(19) United States
(12) Patent Application Publication
(10) Pub. No.: US 2026/0116915 A1
(43) Pub. Date: Apr. 30, 2026

(54) A Chemoselective Photochemical Bioconjugation Reaction and Methods of Use Thereof

(71) Applicant: Trustees of Boston College, Chestnut Hill, MA (US)

(72) Inventors: Abhishek CHATTERJEE, Lexington, MA (US); Soumya Jyoti Singha ROY, San Diego, CA (US)

(21) Appl. No.: 18/856,145

(22) PCT Filed: Apr. 21, 2023
(86) PCT No.: PCT/US2023/066056

§ 371 Date: Oct. 11, 2024

Related U.S. Application Data
(60) Provisional application No. 63/333,593, filed on Apr. 22, 2022.

(51) Int. Cl. C07K 1/107

(52) U.S. Cl. CPC C07K 1/1077

(57) ABSTRACT

Described herein is a visible light enabled photocatalytic oxidative protein bioconjugation reaction directed towards site-specifically incorporating non-canonical amino acids (ncAA).

### RELATED APPLICATIONS

This application claims the benefit under 35 U.S.C. § 119(e) of U.S. Provisional Application No. 63/333,593, filed on Apr. 22, 2022, which is incorporated herein by reference in its entirety.

### GOVERNMENT SUPPORT

This invention was made with Government support under contract number NIH R35 GM136437, awarded by the National Institutes of Health, and contract number NSF 2128185, awarded by the National Science Foundation. The Government has certain rights in the invention.

### REFERENCE TO AN ELECTRONIC SEQUENCE LISTING

The contents of the electronic sequence listing (0342.0013WO1_ST26.xml; Size: 48,138 bytes; and Date of Creation: Mar. 27, 2023) is herein incorporated by reference in its entirety.

### BACKGROUND OF THE INVENTION

Precise site-specific covalent conjugation¹⁻⁸ of native full-length proteins is the enabling technology behind a plethora of important diagnostic and therapeutic modalities, including antigen specific cell type targeted antibody-drug conjugates, nuclear or optical bio-imaging probes, PEGylated biologics, immobilized proteins for enzyme linked immunosorbent assay (ELISA) to name a few.⁹⁻¹¹ Site-specific protein bioconjugation traditionally relied upon nucleophilic natural amino acids, a few venerable examples being lysine labeling with N-succinimidyl ester (NHS) esters, cysteine labeling with maleimide etc. Although operationally simple, targeting canonical amino acid derived nucleophilic residues with electrophilic war-heads often lacks perfect site-specificity, due to the presence of competing reactive sites on proteins leading to suboptimal yield of the desired conjugate. Additionally, the resulting conjugates are often heterogeneous, which compromises their desired activity. Genetically encoded non-canonical amino acid (ncAA) with a bioorthogonal conjugation handle offers an attractive method to achieve perfect site-specific protein labeling. These ncAAs can be co-translationally incorporated into full-length proteins at predefined sites using a repurposed nonsense codon, providing a conjugation-ready site for downstream labeling to prepare homogeneous protein conjugates. Several ncAAs bioconjugation having conjugation handles such as azides, alkynes, aldehyde/ketone, tetrazines, strained alkenes, etc., have been genetically encoded for a variety for producing protein conjugates with unprecedented versatility and precision.^{2,12-15} Nonetheless, there is continued demand for new site-specific protein conjugation strategies that offer unique advantages and are compatible with existing strategies to enable protein labeling at multiple sites with distinct entities.^{16,17}

### SUMMARY OF THE INVENTION

Photochemistry has long played an important role in synthetic organic chemistry, and visible light-enabled photoredox catalysis has experienced a renaissance in the last two decades, spawning countless innovative strategies to unlock novel reactivities. It offers numerous advantages, including mild reaction conditions, and intrinsic spatiotemporal control which are particularly attractive for modifying peptides and proteins. Indeed, photoredox catalysis has been leveraged to develop novel bioconjugation strategies targeting specific canonical amino acid residues, as well as the termini of peptides and proteins. While these examples corroborate the promise of this approach, there are several challenges limiting their practical utility in protein bioconjugation. These include inefficient/incomplete conversion when applied to full-length folded proteins, an intrinsic lack of control over site and stoichiometry of protein labeling when targeting canonical amino acid residues, and off-target reactivity. Development of an efficient and chemoselective photoredox catalyzed reaction that can generate functional site-specific protein conjugates under mild conditions will be a valuable addition in the bioconjugation tool box.

Described herein is a novel visible light-catalyzed conjugation reaction between one, or more, surface-exposed non-canonical amino acids referred to herein as ncAAs, or a non-naturally-occurring amino acids, e.g., 5-hydroxyindoles, or 5-aminoindoles, such as 5-hydroxtryptophan (5HTP), incorporated into a protein, polypeptide or a peptide, and primary anilines that can be used to modify recombinant proteins with excellent efficiency and site-selectivity. The ncAA(s) can be site-specifically incorporated into any recombinant protein—expressed either in bacteria or eukaryotic cells—with high fidelity and efficiency, for example, using an engineered E. coli tryptophanyl-tRNA synthetase (EcTrpRS)/tRNA pair that has been recently developed (see U.S. Pat. No. 10,717,975, the teachings of which are incorporated herein in their entirety). As an example, leveraging its electron-rich nature, 5HTP can be chemoselectively functionalized with anilines, using visible light (blue or red LED; 455 or 650 nm) and methylene blue as the photocatalyst, through a novel oxygen-dependent oxidative coupling reaction. This reaction has been modelled at the small molecule level, and the structure of this unique coupling product has been elucidated. The reaction is referred to herein as the PhotoCLIC reaction and was shown to be compatible with diverse aniline substrates, as well as different substrate proteins including a full length antibody, and it was used to generate a homogeneous, functional fluorophore-nanobody conjugate. Finally, as described herein, the PhotoCLIC is compatible with strain-promoted azide-alkyne cycloaddition, one of the most popular bioorthogonal conjugation strategies, and that these two reactions can be concurrently used to create a homogeneous dual-labeled peptide, polypeptide or a full-length protein.

Specifically described herein are novel photocatalytic methods of oxidative conjugation of genetically encoded non-canonical amino acids (ncAAs). More specifically, described herein is the development of a novel aerobic oxidative conjugation reaction that works with primary aromatic amine derivatives as conjugating agent in the presence of a photocatalyst, for example, methylthioninium chloride (methylene blue). An aromatic amine is an amine in which nitrogen is bonded to one or more aryl groups. Examples of aromatic amines include aniline, or substituted aniline. Other examples of aromatic amines are well known to those of skill in the art. The term for this chemical reaction is referred to herein as “Photoredox-Catalyzed Labeling with Chemoselectivity”, or the “photoCLIC” reaction. Noteworthy, the product of the photoCLIC reaction was found to be different than the known ferricyanide based oxidative bioconjugation reaction²¹. Although there are a number of photoinduced bioconjugation reactions with genetically encoded ncAA,⁴⁻⁴⁷ they typically require irradiation in the UV region (<400 nm) whereas, described herein the data demonstrates the first report of a visible light promoted photocatalytic bioconjugation reaction applicable to a genetically encoded ncAA.

The photoCLIC method described herein represents a photocatalytic “tag and modify”⁶² paradigm for the bioconjugation of native (e.g., wild-type) proteins that should also have applications for late-stage diversification of polypeptides (with a hydroxyindole core introduced through ribosomal or chemical protein synthesis) to access novel chemical space for applications in medicinal chemistry. Additionally, the photoCLIC reaction carries a unique advantage that ncAAs, and in particular, 5-hydroxyindole groups or a 5-aminoindole groups and the coupling partner aromatic amine can both be biosynthetically produced and co-translationally incorporated inside cell^{23,63} (without needing exogenous supply of either) and a productive merger with photoCLIC reaction will produce novel lanthipeptides.⁶⁴ Importantly, the photoCLIC reaction represents the first example of a visible light promoted photocatalytic bioconjugation reaction directed towards genetically encoded surface-exposed ncAAs and would pave the way for development of newer bioconjugation reactions targeting newer ncAAs through photocatalytic activations for the chemical diversification of genetically encoded biopolymers which would have applications in biotechnology, material science and catalysis.^{9, 10, 65, 66}

Encompassed by the present invention is a method of chemoselective modification of a protein or peptide, wherein the protein or peptide comprises one, or more, surface-exposed non-canonical amino acids comprising a 5-hydroxyindole group or a 5-aminoindole group, the method comprising reacting/conjugating the non-canonical amino acid of the protein or peptide with an aromatic amine derivative in the presence of a photocatalyst under conditions suitable to conjugate/covalently couple the aromatic amine derivative to the non-canonical amino acid of the protein or peptide, thereby modifying the protein or peptide in a chemoselective manner. Importantly, as described herein, the photocatalyst is reactive under visible light irradiation in the range of about 455 nm to about 650 nm.

Specifically encompassed herein, are methods incorporating the use of a photocatalyst, for example, selected from the group consisting of: methylthioninium chloride (methylene blue), rose bengal, eyosin-Y, crystal violet, malachite green, flavin derivatives, or bromophenol blue. Other suitable photocatalysts can be determined using the methods described herein. In one embodiment of the present invention, the bioconjugation method is externally controlled by light exposure. Thus, an important advantage of the photoCLIC methods described herein is the ability to remotely control the reaction spatiotemporally through gating the light exposure and usability of the reaction in sequence with other well-established bioconjugation reactions (e.g. strain promoted azide-alkyne cycloaddition) for the preparation of dual site modified proteins.

The methods of the present invention specifically conjugate/couple the target ncAA of the biomolecule in a site-specific manner. That is, the target ncAA is selectively conjugated over other amino acids (such as naturally occurring amino acids) present in the protein, peptide or polypeptide molecule. Specifically, the non-canonical amino acid is chemoselectively oxidized over any other canonical amino acid in the protein or peptide. As used herein the term “chemoselective” refers to the selective and preferential reaction of a chemical reagent that operates exclusively on one functional group in the presence of other functional groups.

The chemoselectivity of the present invention allows for the specific incorporation of additional moieties attached/coupled to or associated with to the aromatic amine derivatives, thereby site-specifically modifying the ncAA of the molecule. Therefore, also encompassed by the present invention are photoCLIC methods wherein the aromatic amine derivative comprises a cargo moiety. The generality of the photoCLIC reaction has been demonstrated herein with a range of aromatic amine derivatives containing useful cargos such as detectable labels, for example, alkyne, biotin and fluorophore, for the site-specific conjugation of different recombinantly expressed proteins/enzymes such as green fluorescent protein, myoglobin, anti-Her2-nanobody, nanoluciferase.

As described herein, present methods chemoselectively label or conjugate targeted non-canonical amino acids genetically incorporated into biomolecules such as proteins or nucleic acids. The non-canonical amino acids can specifically comprise a 5-hydroxyindole group wherein the 5-hydroxyindole group is a 5-hydroxytryptophan (5HTP), or an analog thereof. Also specifically encompassed herein is the non-canonical amino acid comprising a 5-aminoindole group wherein the 5-aminoindole group is 5-aminotyrtophan, or an analog thereof. Other ncAAs can also be conjugated by the methods described herein.

More specifically, the present methods encompass a visible light-enabled photocatalytic oxidative protein conjugation, the method comprising contacting a protein or peptide, wherein the protein or peptide comprises one, or more, surface-exposed non-canonical amino acids comprising a 5-hydroxyindole group or a 5-aminoindole group, with an aromatic amine derivative in the presence of a visible light enabled photocatalyst under aerobic conditions suitable to conjugate the aromatic amine derivative to the non-canonical amino acid of the protein or peptide.

The method of chemoselective conjugation of a protein or peptide described herein can comprise the following steps: a) incorporating one, or more, non-canonical amino acids into the protein or peptide in a site-specific manner; and b) reacting the one, or more non-canonical amino acids of the protein or peptide with an aromatic amine derivative under photocatalytic oxidative conditions suitable to covalently conjugate the aromatic amine derivative to the non-canonical amino acid of the protein or peptide, thereby modifying the protein or peptide in a chemoselective manner. Specifically, the non-canonical amino acid targeted by the conjugation method comprises a 5-hydroxyindole group or a 5-aminoindole group and, more specifically wherein the 5-hydroxyindole group is 5-hydroxytryptophan (5HTP), or an analog thereof, or wherein the 5-aminoindole group is 5-aminotyrtophan, or an analog thereof.

As described herein, five proteins harboring a 5HTP residue at a surface-exposed site were expressed using the EcTrpRS/tRNA pair: sfGFP; myoglobin-99-5HTP; nanoluciferase-2-5HTP; the anti-Her2-nanobody with 5HTP at the site 69 (anti-Her2-nanobody 69-5HTP); and a full length humanized antibody Trustuzumab having its 198 residue of heavy chain (HC mutated to 5HTP. Treatment with the biotin-aniline derivative 9 under PhotoCLIC conditions resulted in efficient single-labeling of the 5HTP In each case, we also expressed the corresponding wild-type protein lacking the 5HTP residue mutants for each of the proteins.

Further encompassed by the present invention is a kit for the chemoselective modification of a protein or peptide, wherein the protein or peptide comprises one, or more, non-canonical amino acids, the kit comprising the following components; a container containing an aromatic amine derivative compound; a photocatalyst; and reagents suitable for the conjugation of the aromatic amine derivative compound to the protein or peptide. The aromatic amine derivative of the kit can further comprise a cargo moiety as described herein.

The photoCLIC reaction described herein represents the first example of a visible light promoted photocatalytic bioconjugation reaction directed towards a genetically encoded ncAA and opens up the possibility of development of newer bioconjugation reactions targeting novel ncAAs through photoredox catalysis for the chemical diversification of genetically encoded polypeptides which would have applications in biotechnology, material science and bio-catalysis.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. The patent or application file contains at least one drawing executed in color. Copies of this patent or patent application publication with color drawings(s) will be provided by the Office upon request and payment of the necessary fee. Of the drawings:

FIGS. 1A-C—Nonsense (TGA) suppression with E. coli derived evolved tryptophanyl-tRNA synthetase (EcTrpRS)/tRNA^{EcTrp} pair can provide recombinant proteins with site-specifically incorporated 5-hydroxy tryptophan (5HTP) both in E. Coli and mammalian cells.19 5HTP (an over the counter drug in the USA) is cheaply available, stable non canonical amino acid (ncAA) that can also be biosynthetically produced within the cell and co-translationally incorporated through non-sense suppression, obviating the need of externally adding it in the cell growth media. (B) The hydroxyindole core of 5HTP can act as amphiphile; naturally hydroxyindole is a potent nucleophile capable of reacting with an external electrophile (e.g. diazonium salts); in the presence of an oxidant (e.g. super stoichiometric potassium ferricyanide) hydroxylindole can react with an external nucleophile (e.g. aromatic amine) possibly through the intermediacy of a radical cation or electrophile. (C) A priori hypothesis of a 5-hydroxyindole directed photocatalytic oxidative conjugation reaction with genetically incorporated 5HTP for protein labelling. 5-hydroxy tryptophan (5HTP) can be site-specifically incorporated into recombinantly expressed proteins in E. coli or mammalian cells through nonsense (TGA) suppression using an engineered E. coli derived tryptophanyl-tRNA synthetase (EcTrpRS)/tRNA^{EcTrp} pair.^{[17]}Selective oxidation of the uniquely electron-rich 5-hydroxyindole sidechain of 5HTP, mediated by an excited state photocatalyst (PC*), may enable its subsequent coupling with a suitable conjugation partner (e.g., anilines).

FIGS. 2A-E (A) Optimized PhotoCLIC conditions enable selective labeling of sfGFP-151-5HTP. Entries 1-9 represents necessary elements of the optimized conditions. ^{a}conversion to product (%) assessed by whole-protein MS analysis. ^{b}Reaction time was 45 min. ° Product MS shows off-target oxidation, ^{d}Product MS shows pronounced off-target oxidation. (B) ESI-MS of sfGFP-151-5HTP labeled with 1 under the optimized PhotoCLIC conditions; no labeling was observed for the wild-type sfGFP control under the optimized PhotoCLIC conditions. (C) LC-MS analysis of the PhotoCLIC reaction modeled at the small-molecule level; the product structure was determined by correlation NMR, MS analysis, and chemical derivatization studies shown in (D). The product may exist in different tautomeric forms; A₂₅₄ represents absorbance at 254 nm. (E) Plausible mechanism for the formation of 3.

FIGS. 3A-C. (B) Generality of the photoCLIC labelling conditions demonstrated through the conjugation of sfGFP-151-5HTP with different aromatic amine derivatives (4-9). (A) structures of the aromatic amine derivatives are shown along with their exact masses. (C) A number of different primary aniline derivatives (4-9) were purchased or synthesized, some appended with useful functionalities such as a terminal alkyne, a biotin, or a short polyethylene glycol (PEG) chain. Each of these substrates conjugated efficiently to sfGFP-151-5HTP under the optimized PhotoCLIC condition to generate homogeneous singly labeled products. (B) In each case, the conjugate mass was consistent with the PhotoCLIC reaction mechanism elucidated above. As expected, these aniline derivatives failed to couple to WT-sfGFP under identical conditions further attesting to the chemoselectivity of the reaction.

FIGS. 4A-B. (A) Bottom-up mass spectrometry of photoCLIC labeled sfGFp-151-5HTP with 1 confirms chemoselectivity of 5HTP for the labeling reaction. All peptides with the 1 modification stem from 5HTP-containing peptide fragment through alternative trypsin digestion. The major peptide is a product of complete trypsin digestion, whereas the less-abundant peptides comes from missed cleavages. B) MS-MS analysis of the most abundant modified peptide confirms 5HTP at the 151 position as the site of modification.

FIGS. 5A-E. (A) PhotoCLIC enables hydoxyindole-selective biotinylation (using 9) of different proteins of interest (POI): (B) anti-her2-nanobody-69-5HTP, (C) myoglobin-99-5HTP, (D) nanoluciferase-2-5HTP and (E) full length Trustuzumab having HC-198-5HTP. Corresponding wild type proteins (lacking 5HTP) showed no labelling under otherwise identical conditions. For starting (unlabeled) and labeled Trustuzumab, LC-MS was done after reductive separation of HC and LC followed by PNGase F digestion to remove the glycan PTM on N297 of HC. Both unlabeled and labeled HC has some undigested glycoform; MB: methylene blue.

FIGS. 6A-C. (A) Fluorophore labeling of anti-Her2 nanobody using PhotoCLIC. (A) MS analysis demonstrates selective labeling of anti-Her2-nanobody-69-5HTP with 5-aminofluorescein, while the corresponding wild-type protein remains unreacted upon identical treatment. (B) SDS-PAGE followed by fluorescence imaging shows PhotoCLIC-mediated fluorophore labeling of anti-Her2-nanobody-69-5HTP, but not the corresponding wild-type protein under identical conditions. (C) FACS analysis demonstrating successful staining of SK-BR-3 cells by fluorophore-labeled anti-Her2-nanobody

FIGS. 7A-B. (A) An sfGFP with site-3 mutated to 5HTP and site 151 mutated to AzK (sfGFP-3-HTP-151-AzK) was recombinantly expressed in E. coli using our dual nonsense suppression technology. (B) Sequential double modification of the resulting protein, first using SPAAC followed by PhotoCLIC, demonstrated by whole-protein MS analysis. BCN derivative: (1R,8S,9s)-Bicyclo[6.1.0]non-4-yn-9-ylmethanol; AzK: NF-2-azidoethyloxycarbonyl-L-lysine.

FIGS. 8A-B. (A) Screening of conjugating partners for 5HTP under photoredox conditions. (B) Structures of coupling partners/reactants.

FIG. 9. A graph of the demonstration of temporal control of the PhotoCLIC labeling through following light ON/OFF cycle: 5 min ON then 5 min OFF, 5 min ON then 5 min OFF, 5 min ON 5 min OFF, 5 min ON, 5 min OFF, 5 min ON.

FIGS. 10A-C. MS analysis of wild-type and 5HTP mutated anti-Her2-nanobody (A), myoglobin (C) and nanoluciferase (B); biotin labeling of these three recombinant proteins using PhotoCLIC. Corresponding wild type proteins showed no labeling under otherwise identical conditions. Amino acid sequences are found herein.

FIG. 11. Selective biotinylation of Trustuzumab HC-198-5HTP with 9 under photoCLIC conditions, revealed by anti-biotin Western blot (using a streptavidin-HRP probe). Both WT-Trustuzumab and Trustuzumab HC-198-5HTP were subjected to biotinylation under photoCLIC conditions, desalted and resolved in SDS-PAGE (reductive denaturing) gel followed by Western blot.

FIGS. 12A-B. Fluorescein labeling of sfGFP-151-5HTP with 10. MS analysis (A), and SDS-PAGE followed by fluorescence imaging (B)) shows selective fluorescein labeling of sfGFP-151-5HTP under PhotoCLIC conditions, but not of WT-sfGFP.

FIGS. 13A-B. (A) Plasmid map of pET22b-T5-nanoluciferase-3-TGA; (B) nucleic acid sequence.

FIGS. 14A-B. (A) Plasmid of pET-22b-Myoglobin-99-TGA (sperm whale myoglobin-99-TGA; (B) nucleic acid sequence.

FIGS. 15A-B. (A) Plasmid map of pET22b-T5-5F7-69-TGA_(anti-Her2-nanobody-69-TGA); (B) nucleic acid sequence.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Described herein is a visible light enabled photocatalytic oxidative protein bioconjugation reaction directed towards site-specifically incorporated non-canonical amino acid (ncAA) such as 5-hydroxytryptophan (5HTP). The reaction requires catalytic amount of methylene blue in the presence of blue or red light emitting diodes (455 nm or 650 nm) and found to be useful for the site specific chemoselective covalent conjugation of full length native proteins having a genetically encoded 5HTP residue using bench-stable aromatic amine derivatives as the coupling reagent within reasonably short reaction times (30-60 min).

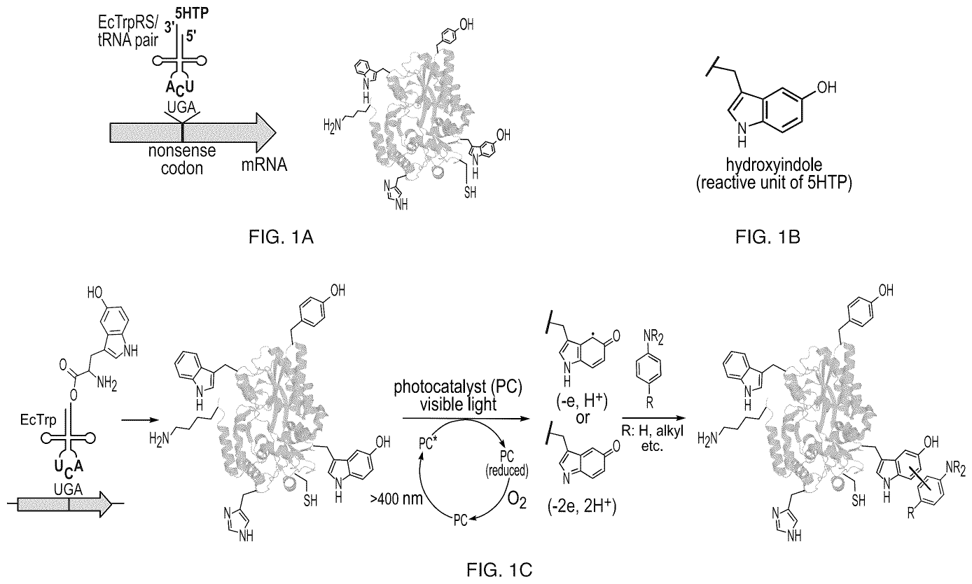

Methods of genetically engineering ncAAs such as 5-hydroxytryptophan (5HTP) in proteins expressed in both prokaryotic and eukaryotic cells have been described¹⁷⁻¹⁹ (FIGS. 1A-C) and new conjugation strategies targeting this unique ncAA for site-specific protein labeling have been developed.^{17,20-22} 5HTP is attractive as an exemplary bioconjugation handle/tag or label, for several reasons: 1) relative to other conjugation-capable ncAAs, 5HTP is drastically less expensive (˜$10/g), 2) 5HTP can be biosynthetically produced in a single step from tryptophan, obviating the need for exogenous supply for it to be co-translationally incorporated,^{23,24} and 3) chemistries targeting 5HTP are intrinsically orthogonal to established bioconjugation strategies such strain-promoted azide-alkyne cycloaddtion (SPAAC)¹⁷ 4) Hydroxyindole, the core moiety of 5HTP, has amphiphilic reactivity capable of reacting with an external electrophile as well as nucleophile (FIGS. 1A-C). However, it is important to note that the methods described herein can be modified for other ncAAs based on the novel methods herein.

The hyper-nucleophilicity of 5HTP was exploited to develop a novel chemoselective rapid azo-coupling reaction (CRACR) for protein bioconjugation.²² Subsequently, an oxidative bioconjugation reaction has been developed through (polarity reversal) activation of 5HTP with potassium ferricyanide as the oxidant.²¹ The attractive advantage of this umpolung oxidative bioconjugation strategy is the use of widely available and bench-stable commodity chemical aromatic amine derivatives as the conjugating partner. Although ferricyanide is a mild oxidizing agent and has been utilized for interesting bioconjugation reactions,²⁵⁻²⁸ the use of over-stoichiometric potassium ferricyanide often causes over-oxidation of the protein conjugate, especially during conjugation of aromatic amines carrying a larger molecular cargo. In addition, it was noted that certain conjugation probes might have compatibility issues with the oxidant and strictly necessary step to use an over-stoichiometric reducing agent (TCEP) to quench the reaction²¹ might compromise the functional potency of certain protein/enzyme conjugates restricting the applicability of the oxidative bioconjugation strategy.

In this context, the principles of catalysis for carrying out novel bioconjugation reactions²⁹ were applied and a catalytic method of oxidation of site-specifically incorporated 5HTP would provide a platform to avoid using super-stoichiometric oxidant and would allow to avoid over stoichiometric reductive quencher was evolved. To this end, the recurrent interest of application of visible light photoredox catalysis³⁰ for the modifications of small peptides,³¹⁻³⁵ and full length proteins³⁶⁻⁴² through canonical amino acid directed conjugation reactions⁴³ led to the hypothesis that one could functionally replace ferricyanide with a photoredox catalysis manifold capable of promoting a catalytic oxidation. Mechanistically, it was surmised that the ferricyanide mediated oxidative bioconjugation²¹ directed to hydroxyindoles (reactive unit of 5HTP) proceeds via the generation of a protein centered radical cation which gets trapped by an aromatic amine derivation.²¹ It was postulated that (owing to the high electron density and consequently low redox potential of hydroxyindole moiety) a suitable photo-catalyst under a photo-excited state would be able to selectively oxidize hydroxyindole residue prior to any other native amino acid side chain; this would generate a radical cation (through one electron oxidation) or an electrophile (through two electron oxidation) embedded in the context of a native protein that can be intercepted by a suitable nucleophile or radicophilic conjugating reagent (FIG. 1C). Particularly, a visible light photoredox system was sought that would emulate the reactivity of potassium ferricyanide with concurrently making the oxidative bioconjugation catalytic as well as amenable to spatio-temporal control. Interestingly, efforts towards this direction led to the serendipitous realization (finding) of a novel visible light promoted chemoselective photocatalytic bioconjugation reaction directed towards a site-specifically incorporated ncAA, 5-HTP.

To replicate ferricyanide based oxidative conjugation²¹ with a photocatalytic system, it was planned to react a super folded green fluorescent protein (sfGFP) with a 151-site mutated to 5HTP^{18,19} (sfGFP-151-5HTP) to a primary aromatic amine derivative (4-aminophenylacetic acid 1) in the presence of different photo-catalysts and suitable visible light irradiation. At the outset, it was important to be cognizant of the following reaction metrics associated with the implementation of a site-selective photocatalytic oxidative bioconjugation reaction: 1) the success would hinge on the chemoselective oxidation of 5HTP over any other canonical amino acids to ensure a chemoselective conjugation reaction; this aspect becomes particularly important in the context of native proteins where a micro-environment (through tuning the local hydrogen bonding network) could make a canonical amino acid (e.g. tyrosine) more electron rich³⁶ and hence more easily oxidizable. 2) Availability of a photocatalyst that can operate under visible light irradiation conditions in an aqueous buffer and is compatible (non-denaturing) to proteins. In this respect common organic dyes used for histological staining (methylene blue, rose bengal, eyosin-Y, crystal violet, malachite green, bromophenol blue) and commonly used organic photoredox catalysts [e.g. Ru(bpy)₃²⁺] were chosen for screening. 3) The conjugating agent should be stable under the photo-redox conditions and ideally does not require an organic co-solvent which might be detrimental to the stability of the native protein; 4) The conjugated product must be stable and should remain functionally active post-conjugation. In this respect, the optimized photocatalytic system should subdue or nullify any potential protein over-oxidation which is one of the major concern associated with light mediated bioconjugation reactions. Additionally, it was desirable to avoid the need of rigorous exclusion of air (that is often a requirement for a photoredox bioconjugation reactions) to have an operationally easier bioconjugation method.

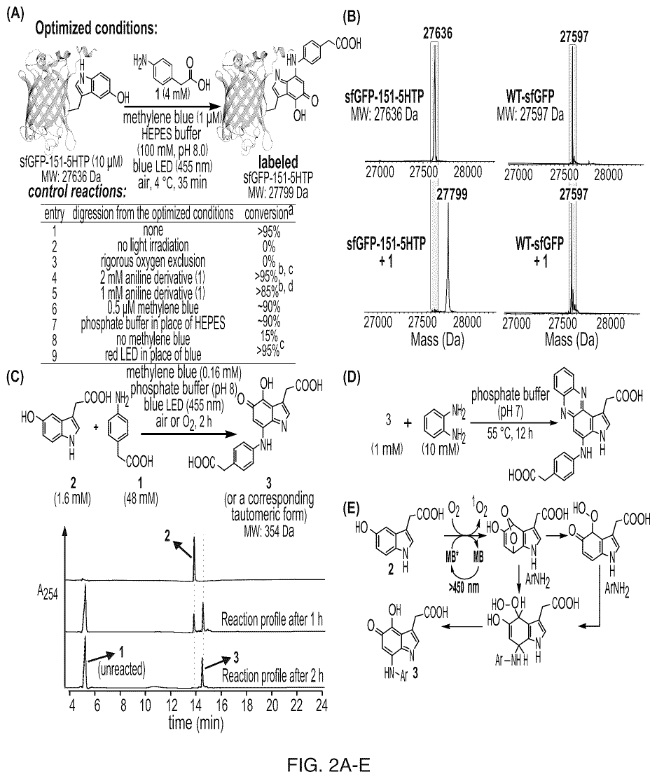

When a mixture of sfGFP-151-5HTP (10 μM) and 4-aminophenylacetic acid 1 (4 mM) in phosphate buffer (pH 8) was subjected to blue LED light irradiation (455 nm, 7 W) in the absence of any photo-catalyst for 35 min, surprisingly ˜10% starting protein was converted to a labelled product having a mass of 27799 Da. (See FIGS. 2A-E). Formally, the mass of the product represented a species having a 12 Da mass addition to the combined mass of the sfGFP-151-5HTP (mass: 27636 Da) and 4-aminophenylacetic acid (mass: 151 Da) which was evidently a different product than that obtained with ferricyanide mediated oxidative conjugation²¹ reaction (2 Da reduced mass from the combined mass of the protein and aromatic amine derivative). Intriguingly, it was observed that different photo-catalysts could accelerate the reaction while used in sub-stoichiometric amount (1 μM). After a brief optimization, methylene blue was turned out to be the best photo-catalyst in the presence of aqueous HEPES buffer (100 mM) at pH 8.0 at 4° C. providing a clean (>95%) conversion by ESI-MS. Mentionable, riboflavin sodium phosphate, rose bengal and eyosin-Y were also found to be active catalysts showing 70-90% conversion, but with higher level of oxidative protein damage. Although full conversion (>95%) was obtained even with 2 mM 4-aminophelyacetic acid in HEPES buffer, use of 4 mM aromatic amine derivative was found to subvert the protein oxidative damage while keeping the reaction time short. It is important to note that methylene blue is primarily a red absorbing dye (λₘₐₓ 664 nm in 100 mM HEPES buffer), having a marginally reduced (tail) absorption in the blue region. Although, the conjugation reaction can be carried out under red light irradiation conditions (vide infra) blue LED (455 nm) was chosen, as counterintuitively⁴⁸ red light (650 nm LED) showed a slightly higher degree of protein oxidation peaks in the conjugated product. Also, the reaction strictly requires oxygen and it was observed that for the full conversion of 1 μM starting protein dissolved oxygen was sufficient. Interestingly, when the same conjugation reaction was conducted with a wild type sfGFP (having a tyrosine at the 151 site instead of sfGFP-151-5HTP) no trace of aromatic amine conjugation was observed underscoring the chemoselectivity of the optimized (photoCLIC) reaction conditions and provided the impetus to explore the product structure as well as the scope of the novel protein bioconjugation reaction. To elucidate the structure of the product, 5-hydroxyindoleacetic acid (5HIAA) was chosen as a model and it was reacted with 4-aminophenylacetic acid (4-AmPhAcOH) under a reaction condition mimicking the (photoCLIC) protein conjugation conditions. ESI-MS coupled liquid chromatography (LC-MS) of the crude reaction mixture showed the major product to have the m/z 355. Considering 355 to be the singly protonated state of the molecular ion peak, the product should have a mass of 354 Da which is 12 Da addition to the combined mass of the starting materials (191 Da+151 Da+12 Da) reflecting the outcome of the protein modification. Also, the model reaction, akin to the protein modification reactions was found to be strictly oxygen and light dependent and the absence of either stalled the conversion. The structure of the product was assigned by a combination of mass spectrometry, co-relation NMR and product derivatization study. Noteworthy, although there is a report of photo-conjugation of 4-hydroxyindole with aromatic amine (under catalyst free conditions with reaction time in weeks)⁴⁹ to provide a product with similar mass distribution as obtained in the present study, no such aerobic oxidative conjugation pertaining to 5-hydroxyindole is reported.

It is important to note that the conjugation product can stay in different isobaric tautomeric forms⁵⁰ depending on the buffering system and availability of intra- or intermolecular hydrogen bond donor/acceptor etc. The formation of product 3 can be explained through the intermediacy of an endoperoxide intermediate generated through the reaction of the hydroxyindole with singlet oxygen. Similar endoperoxide species have been implicated as the intermediate in a number of reactions pertaining to the electron rich aromatic systems with oxygen under photochemical conditions.^{37,51-56} For the wild type sfGFP, although no conjugation reaction was observed, approx. 20% starting protein was found to undergo oxidation. The oxidation presumably happened to the surface exposed histidine residues of the C-terminal hexahistidine tag (used for the purification of the recombinantly expressed protein) as histidine is known to undergo singlet oxygen mediated oxidation⁵² but have been reported to be unreactive towards aromatic amine.³⁷

A major advantage of a light promoted bioconjugation reaction is the ability to remotely control the reaction through the control of light exposure. The quest for the same led to experiments to probe the temporal resolution of the photoCLIC reaction. It was found that the photoCLIC reaction can be externally gated by controlling the light exposure, the reaction (conversion to the labelled product) stops if interrupted the light exposure and could be jump-started with resuming the exposure of the light.

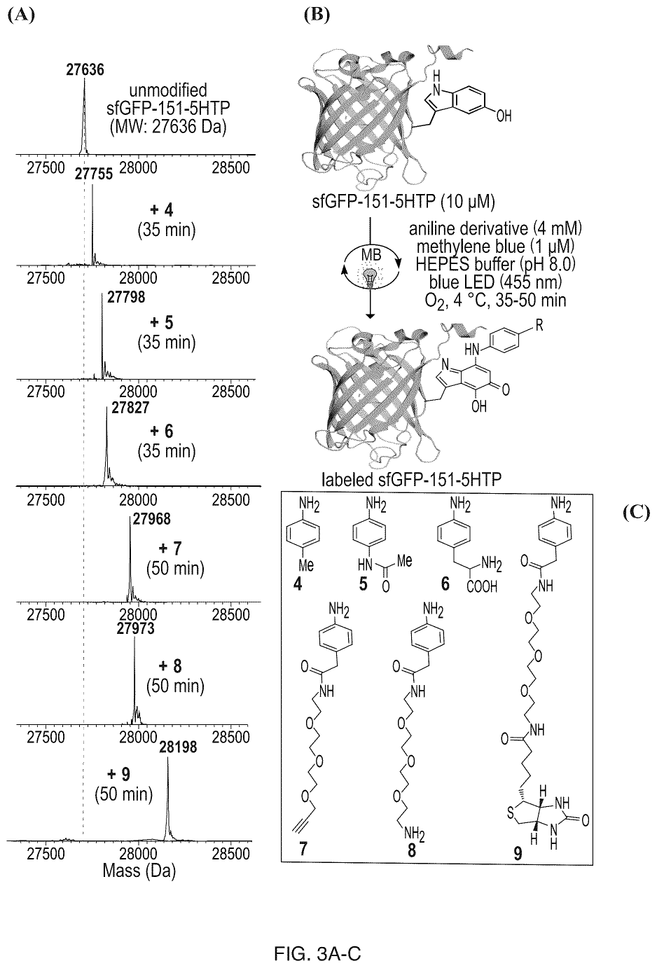

To explore the scope of the photoCLIC reaction, a number of different primary aromatic amine derivatives (4-9) were reacted to sfGFP-151-5HTP to get clean conjugation to singly labelled products (FIGS. 3A-C). This includes conjugation with useful alkyne containing aromatic amine (7) and biotin-aromatic amine (9) derivatives. Notably, for all the aromatic amine conjugates the signature mass of 12 Da addition to the combined mass of protein and the aromatic amine (mass of protein+mass of aromatic amine derivative+12 Da) was observed. Importantly, subjecting the aromatic amine derivatives to wild type sfGFP under photoCLIC conditions seldom showed any conjugation product attesting to the chemoselectivity of the reaction. Interestingly, a number of other nucleophiles (e.g. benzylamine, phenyl hydrazine, phenylhydrazide, N-methylaromatic amine, N, N-dimethyaromatic amine etc.) were found to be unreactive under the optimized conditions.

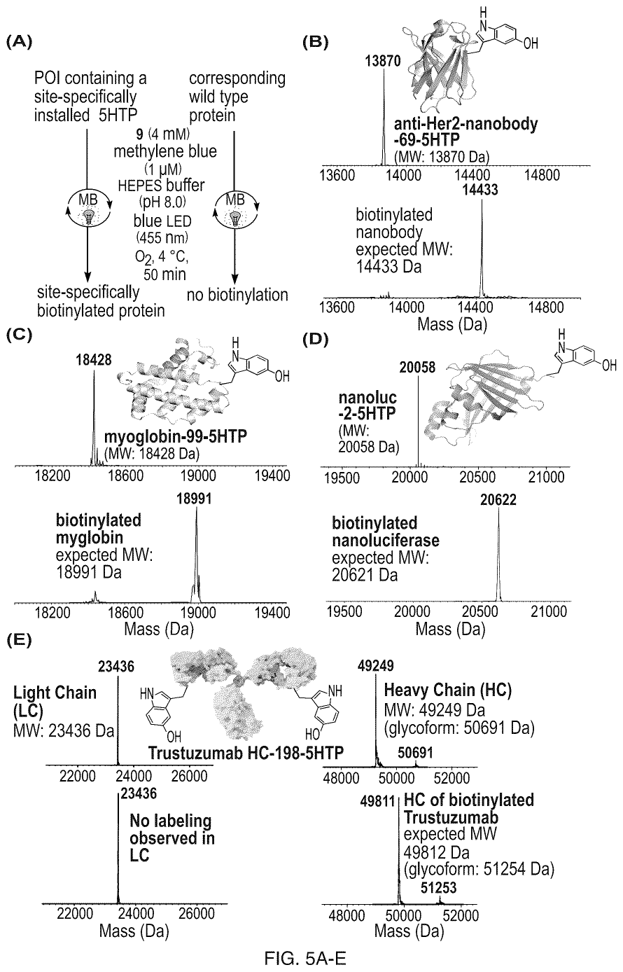

Having established the photoCLIC labelling with sfGFP-151-5HTP it was sought to establish the generality of the conditions with other proteins. The following three proteins with 5HTP at a chosen site were expressed through non-sense suppression: myoglobin with 5HTP at the site-99 (myoglobin-99-5HTP), therapeutically important protein anti-her2-nanobody⁵⁷ with 5HTP at the site-69 (anti-Her2-nanobody 69-5HTP) and nanoluciferase enzyme with 5HTP at the site-2 (nanoluciferase-2-5HTP)(See FIGS. 5A-E). To accomplish this, protein coding sequence with a opal (TGA) stop codon at the chosen site was expressed in a ATMW-BL21 strain¹⁸ of E. coli with a TGA-suppressing tryptophanyl-tRNA synthetase (TrpRS)/tRNA^{Trp} pair¹⁹ with 5HTP in the growth medium. The sites were chosen to have the 5-hydroxyindole (of 5HTP reside) on the surface of the protein and hence available for conjugation. For myoglobin and anti-Her2-nanobody, the sites for TGA codon introduction (and for subsequent 5-HTP incorporation) were determined analyzing the crystal structures of related proteins. For a related myoglobin (PDB ID: 1MCY) site-99 resides in a relatively flexible loop connecting two α-helices.

For anti-Her2-nanobody (for which no crystal structure available), a related crystal structure of anti-GFP nanobody (PDB ID: 3OGO) was used to determine site-69 to be surface exposed as well not near to the complementarity determining region. For introducing 5-HTP to nanoluciferase a TGA codon was genetically added near the N-terminus at the site-2. The mutant proteins were purified with a C-terminal polyhistidine tag by immobilized metal ion-affinity chromatography and successful incorporation of 5HTP was confirmed by whole protein mass spectrometry analysis. The corresponding wild type proteins (without 5HTP) was expressed to carry out control reactions. When these three 5HTP containing proteins were reacted to the biotin-aromatic amine derivative under photoCLIC conditions, singly biotinylated proteins were obtained with good conversions. Expectedly, corresponding wild type proteins did not show any labelling under the same conditions.

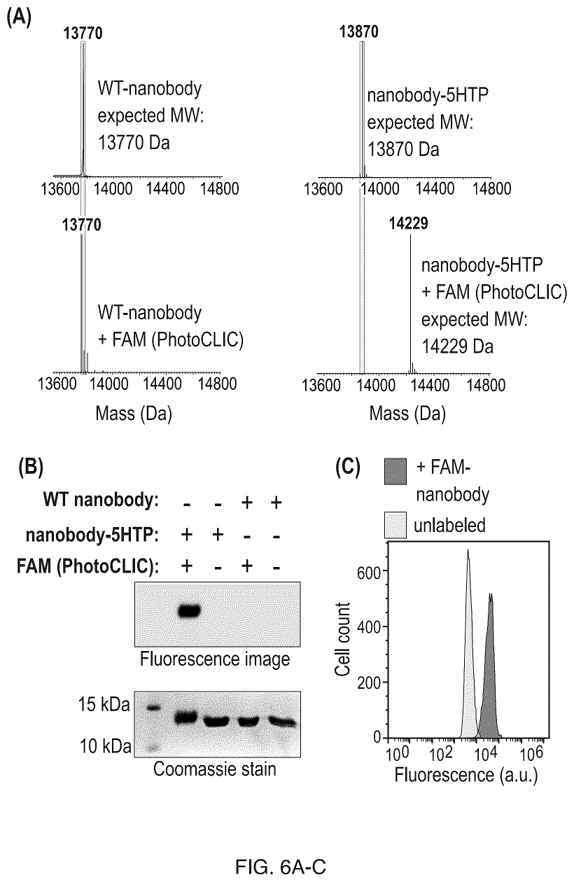

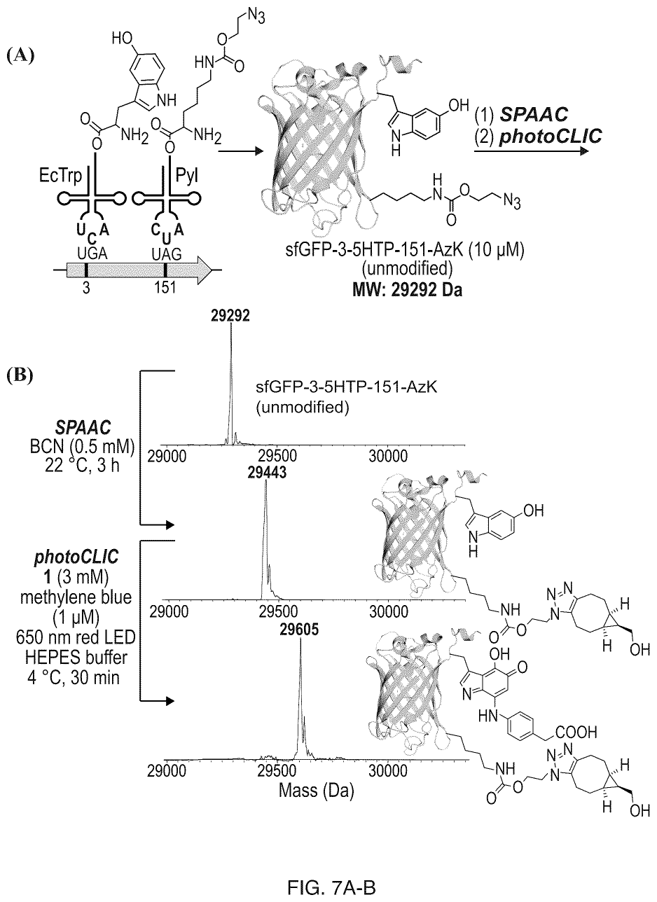

Next, applicability of the conjugation reaction to attach useful biophysical probes to proteins was investigated. As a benchmark study, it was desirable to apply the photoCLIC reaction for fluorophore labelling of proteins using aromatic amine modified fluorescein derivatives as fluorescein derivatives are relatively less expensive and widely used fluorophores for different biophysical and biochemical studies. To this end, it was important that the potential photo-bleaching of the fluorophore with blue light as fluorescein derivatives can absorb blue light. To avoid unwanted photo-excitation and associated photobleaching of the chosen fluorophore, the replacement of the blue LED (455 nm) with a red LED (650 nm) was successfully carried out with the photoCLIC reaction with a fluorescein-aromatic amine derivative. It was found that the anti-Her2-nanobody-69-5HTP could be cleanly labelled with a commercially available aromatic amine containing fluorophore 5-aminofluorescein (10) in the presence of red light (650 nm) under otherwise standard photoCLIC conditions (FIG. 6A). Encouraged by this, an aromatic amine derivative containing carboxyfluorescein (FAM), a fluorescein derivative having much brighter emission characteristics than 5-aminofluorescein, was synthesized. A clean FAM labelling with the aromatic amine-FAM reagent under photoCLIC conditions with sfGFP-151-5HTP was obtained as evident by the ESI-MS analysis (FIG. 6B) and in-gel fluorescence assay (FIG. 6C). Expectedly, corresponding wild type proteins showed no fluorophore labelling (FIGS. 6B and C).

Next, it was investigated if the protein conjugates prepared under the photoCLIC conditions using FAM-aromatic amine remained functionally active. The wild type as well as HTP containing anti-Her2-nanobody was subjected to photoCLIC conditions with FAM-aromatic amine. After getting rid of the excess dye through desalting both the wild type and mutant samples were analyzed by in-gel fluorescence assay to find no fluorescence in the case of wild type protein but fluorescence band in the place of HTP containing nanobody. Most importantly, fluorescence-activated cell sorting (FACS) analysis showed that the FAM labelled anti-Her2 nanobody maintained its functional binding ability to SK-BR-3 breast cancer cells which over-expresses Human epidermal growth factor receptor-2 (Her-2) receptor. HEK-293T cells which does not over-express Her2 receptor on its cell surface, showed marginal cell staining compared to SK-BR-3 cells with FAM labelled anti-Her2-nanobody. This experiment demonstrates the applicability of photoCLIC reaction to prepare functionally active protein conjugates.

Precise dual site modification of a protein has a multitude of applications, for example, the technology can be used for two-color labelling of proteins, capturing protein-protein interactions (PPI), attaching two distinct cargos to an antibody etc.⁵⁸⁻⁶⁰ Technologies have been reported to genetically encode two as well as three bioconjugation handles with 5HTP being one of the ncAA through the development of mutually orthogonal non-sense suppression system.^{17,59,61} It was important to determine if the photoCLIC reaction directed towards 5HTP could be used for the dual site specific derivatization of proteins in sequential manner with a well-established bioconjugation reaction. Gratifyingly, an sfGFP with site-3 mutated to 5HTP and site-151 mutated to AzK (sfGFP-3-5HTP-151-AzK) obtained through dual noncanonical amino acid mutagenesis technology^{17,59} could be sequentially modified with azide reactive strained cyclooctyne derivative (BCN alcohol) through SPAAC (stain-promoted azide-alkyne cycloaddition) reaction followed by the photoCLIC reaction with 4-aminophenylacetic acid to generate precisely dual site modified protein (FIG. 6). This sequential double labelling underscores the compatibility of SPAAC product with the photoCLIC conditions and provides newer opportunities to prepare protein based biomaterials through precise double modification of proteins.

The serendipitous finding and development of a novel photocatalytic method of oxidative bioconjugation directed towards genetically encoded 5HTP has been described herein. The reaction was applicable to the chemoselective site-specific modification of recombinantly expressed proteins having a 5HTP residue utilizing bench stable coupling partner aromatic amine derivatives carrying useful cargos like alkyne, biotin or fluorophore. The photoCLIC reaction can be applied for the sequential double labelling of proteins in conjunction with other bioorthogonal reactions (for, e.g. strain promoted azide-alkyne cycloaddition). The reaction provides an advantage of controlling it remotely through gating the light exposure. The spatiotemporal controllability would provide the opportunity to explore advanced applications of the methodology, for example, for the spatially controlled patterned immobilization of enzymes, antibodies or even whole cells (having 5HTP engineered into the cell surface proteins).

### EXAMPLES

### Example 1: General Protocol for PhotoCLIC Labeling

A solution of 1 μM sfGFP-151-5HTP (or WT-sfGFP), aniline derivatives (4 mM) and methylene blue (1 μM) in HEPES buffer (100 mM, pH 8.0) was irradiated for the required amount time (35-50 min) with blue LED (7 W) in a cold room (4° C.). The resulting mixture was desalted with Bio-Spin® P-6 Gel Columns (Bio-Rad 7326002) pre-equilibrated with 100 mM phosphate buffer (pH 7.4) or using centrifugal filter (10 kDa molecular weight cutoff, Amicon Ultra-0.5 mL) followed by buffer exchanging with phosphate buffer.

The same protocol was followed for PhotoCLIC labeling of myoglobin-99-5HTP, nanoluciferase-2-5HTP and anti-Her2-nanobody 69-5HTP. For labeling of the antibody phosphate buffer was used and the rection time was 25 min. Corresponding wild-type proteins were also subjected to the PhotoCLIC conditions as controls in parallel.

For fluorophore labeling of anti-Her2-nanobody 69-5HTP, red LED (650 nm) was used and resulting conjugates were desalted by HisPur Ni-NTA resin (Thermo Scientific) and then concentrated using centrifugal filters (3 kDa MWcut-off, Amicon Ultra-0.5 mL) followed by buffer exchanging with phosphate buffer. The desalted FAM-aniline (10) conjugate was used for cell-staining.

Protein conjugates were characterized by whole protein mass spectrometry analysis in a HPLC-coupled ESI-MS system. Fluorescent labelled proteins were analyzed by SDS-PAGE followed by in-gel fluorescence imaging (ChemiDoc MP Imaging System, Bio-Rad) and Coomassie staining.

The PhotoCLIC labeling of proteins can be performed with equal efficacy in a HepatoChem photoreactor (picture shown below) keeping water bath temperature at 4° C.

Oxygen-free control reaction: The components of the PhotoCLIC labeling reaction was reconstituted inside a water-compatible glove box filled with nitrogen by dissolving the reaction components in a deoxygenated double deionized water. The tubes containing the reaction mixture were sealed and were taken out, irradiated with blue LED at 4° C. for 35 min and analyzed by whole protein MS after desalting.

### Example 2: Photoclic Reaction of ˜1.1 Mg Sfgfp-151-5Htp

A solution of ˜1104 μg sfGFP-151-5HTP, 4-aminophenylacetic acid (3 mM) and methylene blue (1 μM) was prepared in 5 mL HEPES buffer (100 mM, pH 8). The solution was split equally to eight parts in 2 mL Eppendorf tubes (˜625 μL each), cooled on ice and placed simultaneously under blue LED in a cold room. After irradiating for 35 min, mixtures from the tubes were pooled together, desalted using centrifugal filter (10 kDa molecular weight cutoff, Amicon Ultra-15 mL) and buffer exchanged with phosphate buffer. The desalted protein was directly analyzed by LC-MS to find complete (>95%) labeling.

Probe 1 labeled sfGFP-151-5HTP (100 μg) was precipitated by adding 10× volume of acetone, vortexed vigorously and then incubated at −20° C. overnight. The sample was centrifuged (15,000 rcf, 10 minutes, 4° C.) and the supernatant was removed. The protein pellet was washed with cold acetone (500 μL) and pelleted by centrifugation (5,000 rcf, 10 minutes, 4° C.). The acetone was removed and the protein pellet was allowed to air dry. The protein pellet was resuspended in 8M Urea/PBS (30 μL) and then 100 mM ammonium bicarbonate (70 μL) was added to a total volume of 100 uL. Reductive alkylation was performed with 50 mM TCEP (5 μL, 15 min, 65° C.) and 500 mM iodoacetamide (2.5 μL, 30 min, 25° C.). The sample was then diluted in PBS (120 μL) and tryptically digested overnight at 37° C. (4 μL of 20 μg diluted in trypsin resuspension buffer) and 100 mM CaCl₂ (2.5 μL). After quenching with 10% formic acid, the tryptic peptide sample was desalted using a SEP-Pak desalting columns (Waters), dried by speed-vac, and resuspended in Buffer A (200 μL, 100% H₂O, 0.1% formic acid) for LC-MS/MS analysis.

LC-MS/MS analysis was performed on an Orbitrap Exploris 240 mass spectrometer running Xcalibur v4.4 (Thermo Scientific) coupled to a Dionex Ultimate 3000 RSLCnano system. Samples (5 μL) were injected directly onto a 4 cm Acclaim PepMap 100 C-18 column and peptides were eluted onto an Acclaim PepMap RSLC. Peptides were separated with a 1-hour gradient from 5% to 25% of Buffer B (20% H₂O, 80% MeCN, 0.1% formic acid) in Buffer A (100% H₂O, 0.1% formic acid) at a flow rate of 0.3 μL/min. The spray voltage was set to 2.1 kV. One full MS1 scan (120,000 resolution, 350-1800 m/z, RF lens 65%, AGC target 300%, automatic maximum injection time, profile mode) was obtained every 2 secs with dynamic exclusion (repeat count 2, duration 10 s), isotopic exclusion (assigned), and apex detection (30% desired apex window) enabled. A variable number of MS2 scans (15,000 resolution, AGC 75%, maximum injection time 100 ms, centroid mode) were obtained between each MS1 scan based on the highest precursor masses, filtered for monoisotopic peak determination, theoretical precursor isotopic envelope fit, intensity (5e4), and charge state (2-6). MS2 analysis consisted of the isolation of precursor ions (isolation window 2 m/z) followed by higher-energy collision dissociation (HCD) (collision energy 30%). The tandem MS data was analyzed by the Thermo Proteome Discoverer V2.4 software package and searched using the SequestHT and Percolator algorithms against a UniprotKB database of E. coli K12 proteome amended to include the sequence for 151-W-sfGFP. Trypsin was specified as the protease with a maximum of 2 missed cleavages. Peptide precursor mass tolerance was set to 10 ppm with a fragment mass tolerance of 0.02 Da. Oxidation of tryptophan (+15.995), 1-labeled of oxidized tryptophan (+179.022), oxidation of methionine (+15.995) as well as acetylation (+42.011) and/or methionine-loss (+131.040) of the protein N-terminus were set as dynamic modifications. Cysteine alkylation (+57.021) was set as a static modification. The false discovery rate (FDR) for peptide identification was set to 1%. All mass-spectrometry data were collected as two technical replicates each from two independent biological replicates.

### Example 3: Site-Specific Dual Labelling of Sfgfp-3-5Htp-151-Azk

sfGFP-3-5HTP-151-AzK (10 μM) reacted with BCN-OH (0.5 mM) for 3 h at the room temperature. The labeled protein was desalted and buffer exchanged to 100 mM HEPES (pH 8.0) using a centrifugal filter (10 kDa cutoff, Amicon Ultra-0.5 mL). The resulting protein in HEPES buffer was mixed with 1 (3 mM) and methylene blue (1 μM) and irradiated for 30 min with red LED (650 nm) in a cold room (4° C.). Protein samples after each step were analyzed by whole protein mass spectrometry analysis using an HPLC coupled ESI-MS.

Protein concentration was determined by absorption measurement using NanoDrop 2000 spectrophotometer (Fisher Scientific ND2000)

### Example 4: Protein Expression and Purification

Expression of sfGFP-151-5HTP, myoglobin-99-5HTP, nanoluciferase-2-5HTP and anti-Her2-nanobody 69-5HTP were performed with ATMW-BL21 strain of E. coli as previously described.^{[67]}

ATMW-BL21 strain harboring a pET22b-T5lac plasmid encoding the protein (with C-terminal hexahistidine tags) and a pEVOL-tacl-EcTrpRS-h13-leuV-tRNA_{UCA} suppressor plasmid was grown at 37° C. to an OD₆₀₀ of 0.6. At this point, protein expression was induced by adding 1 mM IPTG and 1 mM 5HTP was also added. After growing the cultures for 16 h at 30° C. with shaking, cells were harvested by centrifugation (5,000 rcf, 10 min, 4° C.), lysed using B-PER Bacterial Protein Extraction Reagent (Thermo Scientific) supplemented with Halt Protease Inhibitor Cocktail (Thermo Scientific) and 0.01% Pierce Universal Nuclease (Thermo Scientific). Proteins were isolated from clarified lysate using HisPur Ni-NTA resin (Thermo Scientific) following manufacturer's protocol. Protein purity was confirmed by SDS-PAGE analysis and protein molecular mass was characterized using HPLC-coupled ESI-MS of intact proteins.

To express sfGFP-3-5HTP-151-AzK, ATMW-BL21 cells were co-transformed with a pGTEV-3TGA-151TAG plasmid, and pEvoltac-EcTrpRS-h14-MbPylRS suppressor plasmid encoding both EcTrp and the pyrrolysyl pairs.^{[3]} The sfGFP double mutant protein was expressed using 1 mM 5HTP and 1 mM AzK after IPTG (1 mM) induction. The protein was purified and analyzed and desalted as described above.

### Example 5: Expression and Mass Spectrometry Analysis of Trastuzumab-HC-198-5HTP

Expi293 cells were cultured following manufacturer information (ThermoFisher) in Expi293 medium supplemented with 0.5× antibiotic-antimyotic (Thermo Fisher, 15240062) at 37° C. in the presence of 8% CO₂ in sterile Erlenmeyer flasks (plastic) with ventilated caps on a CO₂-resistant orbital shaker (125 rpm). Cultures were prepared by seeding 0.3 million viable cells/mL. The cultures are maintained and expanded every 4^{th} day as they reached a density of 3-5 million viable cells/mL. Cell density and viability were measured using the trypan blue exclusion method using an automated cell counter (Bio-Rad TC20™)

PEI-based transfection method was used for antibody expression. Cells (3-5 million viable cells/mL, minimum of 95% viability) were centrifuged at 200 rcf for 4 min and resuspended in fresh media to 20 million viable cells/mL. To the cultures were added a 1:1 ratio of pCDNA3.1-HC-198-TGA-Trastuzumab and pAcBac-EcWRS-H14-1×-EcWtR(TGA) at a final concentration of 25 g/mL. PEI (linear, 40 kDa, 40 mg/ml initial conc.) was added to a final concentration of 50 g/ml. Cells were incubated with shaking under standard expansion conditions for 3 hours, then diluted to 3-4 million viable cells/mL, and sterile (0.22 m filtered) 100 mM stocks of 5HTP and valproic acid were added to 1 mM and 2 mM final concentrations, respectively. After continuing the expression for 10 days, Expi293 supernatant was harvested by pelleting cells at 800 rcf for 8 minutes and the supernatant was decanted and sterile filtered (0.22 m filter). The media was adjusted to pH 5.4 with 50 mM final conc. of NaOAc. After equilibrating 1 mL Pierce™ Protein G resin (Thermo Scientific, 20398) with 15 column volumes of wash buffer (pH 5.4, 50 mM NaOAc), the filtered media was passed through by gravity flow (300-600 mL). Protein G resin was washed with 50 mM NaOAc (pH 5.4) using a volume equal to approximately half the volume of added media (150-300 mL). Purified antibody was eluted using 100 mM glycine buffer (pH 2.7) and ˜1 mL fractions were collected in tubes containing 120 μL sodium phosphate buffer (1 M, pH 7.4) for immediate neutralization. Fractions containing protein were pooled and was concentrated to ˜1-2 mg/mL final protein desalted and concentrated using Amicon® Ultra 15 (Millipore Sigma UFC901024) centrifugal filters. Concentrated antibody was buffer-exchanged using a PD midiTrap G-25 column (GE Healthcare 28918008) pre-equilibrated with 20 column volumes of PBS. Protein concentration was determined by absorption measurement using a NanoDrop 2000 spectrophotometer (Fisher Scientific ND2000). Purity and conjugation reaction of the protein was determined by HPLC-coupled ESI-MS of fully reduced antibodies.

The antibody sample (˜20 μg) was adjusted to pH 7.0 with phosphate buffer and reduced with 30 mM fresh DTT at 55° C. for 10 minutes. After cooling to 37° C., the reduced antibody sample was incubated with 1 μL Remove-iT® PNGase (New England Biolabs, P0706S) for 60 minutes. To remove PNGase, digested antibody was incubated with 25 μL of settled chitin resin (New England Biolabs, S6651L) on an orbital shaker for 15 minutes. The supernatant was carefully removed by spinning down at 15,000 rcf for 4 minute. ˜250 ng of protein was used for HPLC-coupled MS analysis.

### Example 6: Anti-Biotin Western Blot with Biotinylated Trustuzumab

WT or 5HTP mutant antibody after subjecting to photoCLIC labeling were desalted and then reduced (incubating in ˜30 mM DTT at 55° C., followed by heating at 99° C. for 3 mins after adding reducing Lamelli buffer). The samples were resolved in a 12% SDS-PAGE gel. The resolved proteins were transferred from the gel to a PVDF membrane (Life Technologies) using a Trans-Blot Turbo Transfer System 15 (Bio-Rad; semi-dry blotter, 20 V, 30 min). The membrane was blocked with 2% BSA in TBS-T (25 mM Tris, 137 mM NaCl, 3 mM KCl, 0.1% Tween-20, pH 8.5) at the room temperature (22° C.) with gentle agitation for 1 h. The blocking solution was removed, and membrane was incubated with Streptavidin-HRP (Pierce; at 1:2500 dilution, 10% BSA in TBS buffer was used as the diluent) in 10% BSA for 1 hour with gentle shaking at room temperature. The membrane was washed ˜15 times (15-minute incubation with agitation) with TBST buffer. The membrane was developed using SuperSignal West Dura Kit (Fisher Scientific) and incubated for ˜1 minute before signal detection by the ChemiDoc MP imaging system (BioRad).

### Example 7: Flow Cytometry

After reaching confluence, adherent cultures of SK-BR-3 and HEK293T cells were detached from the plate surfaces using 0.25% trypsin (HyClone) for 2 minutes at 37° C. Trypsin was quenched with two volumes of ice-cold DMEM (having 10% FBS), centrifuged at 2,000 rcf for 5 min, then re-suspended in ice-cold PBS at 1-2 million cells/mL. Cells were treated with 100 nM nanobody-69-5HTP-flourescein-aniline (11) conjugate and gently rocked for 15 minutes at 4° C. Cells were washed with and resuspended in ice-cold PBS, then analyzed by flow cytometry on a BD Accuri C6 Plus flow cytometer (BD Biosciences) using the FITC settings (excitation wavelength: 488 nm, standard filter: 533/30). 10,000 events were collected in BD Accuri C6 Plus. Version 1.0.23.1, and data were processed using FlowJo, version 10.8.1.

### Example 8: Small Molecule Synthesis and Characterization

Unless mentioned otherwise, all reactions were carried out with anhydrous solvents (source: Fisher Scientific) under an N₂ atmosphere in oven dried glassware. All work up and purifications were carried out under ambient conditions and with reagent grade solvents. 5-hydroxy-L-tryptophan (5HTP) and 5-hydroxyindoleacetic acid was obtained from Chem Impex International and Alfa Aeser, respectively. Room temperature refers to 22° C. Thin-layer chromatography was performed using Merck silica gel 60 F254 pre-coated plates (0.25 mm). NMR spectra were recorded on Varian INOVA 500 MHz or 600 MHz NMR spectrometers at 25° C. NMR data were processed using Mnova (Mestrelab). Chemical shifts are reported in ppm from tetramethylsilane (δ 0.00) with the solvent resonance as the internal standard. For ¹H-NMR, data are reported as follows: chemical shift, multiplicity (s=singlet, d=doublet, dd=double doublet, q=quartet, t=triplet, m=multiplet), coupling constants (Hz) and integration. Unless mentioned otherwise, reactions were monitored by liquid chromatography (Agilent Technologies 1260 Infinity) coupled mass-spectrometry (Agilent Technologies 6230 TOF LC/MS) using a reverse phase analytical column (Phenomenex) with mobile phase comprises of water-acetonitrile-0.1% formic acid. Reverse phase HPLC (RP-HPLC) purifications were performed in a Waters HPLC system (fitted with a Waters 1525 binary pump and Waters 2489 UV-VIS detector); mobile phase for the RP-HPLC purification comprised of solvent-A (water-MeCN-0.1% trifluoroacetic acid in 95:5:0.1 ratio) and solvent B (MeCN-water-0.1% trifluoroacetic acid in 95:5:0.1 ratio).

### Modelling of the PhotoCLIC Reaction:

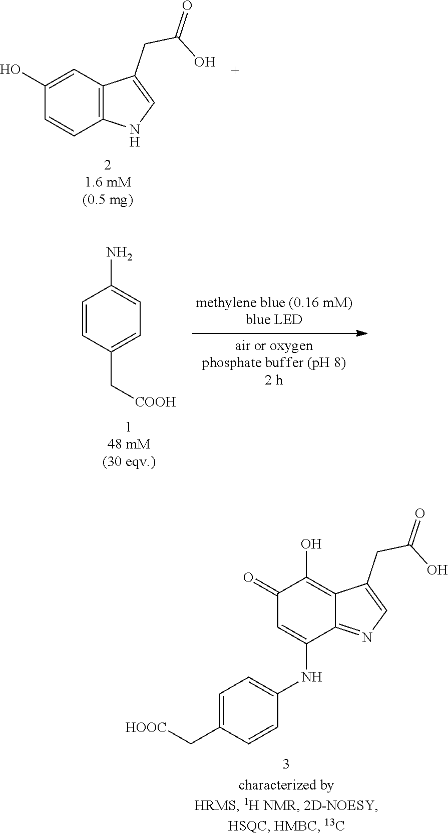 [SMILES: O=C(O)Cc1c[nH]c2ccc(O)cc12 ; Nc1ccc(CC(=O)O)cc1 ; O=C(O)CC1=CN=C2C(Nc3ccc(CC(=O)O)cc3)=CC(=O)C(O)=C12]

5-hydroxyindoleacetic acid (15 mg, 0.078 mmol, 1 eqv.), 4-aminophenylacetic acid (355 mg, 2.35 mmol, 30 eqv.) and methylene blue (2.5 mg, 0.0078 mmol, 0.1 eqv.) were dissolved in 50 mL phosphate buffer (100 mM, pH 8). The total mixture was split into of 15 fractions (each ˜3.4 mL) in 10 mL transparent glass vials. Each of the reaction cocktail was stirred under air or oxygen atmosphere at 22° C. for 2 h under blue LED (18 W, 450 nm, 34 mW/cm², HepatoChem Inc.). After all the 15 batches were reacted, the crude reaction mixtures were pooled, filtered through Chromafil PTFE syringe filters (0.25 mm; pore size: 0.45 m) and the filtrate was purified in multiple batches by reverse phase HPLC using a preparative C-18 column. Fractions with the expected product was concentrated and re-purified by another round of RP-HPLC to get rid of trace 1. Analytically pure 3 (˜15 mg) was obtained as a beige colored solid. Following elution conditions was used for the purification: 0-10 min 100% A, 10-42 min 100% A to 100% B, 42-50 min 100% B, 50-54 min 100% B to 100% A, 54-60 min 100% A; flow rate: 20 mL/min); retention time of the product=23.5 min. ¹H-NMR (600 MHz, D₂O, pD 8.0) δ 7.27 (d, J=12 Hz; 2H), 7.03 (d, J=12 Hz; 2H), 6.80 (s; 1H), 5.46 (s; 1H), 3.55 (s; 3H), 3.48 (s; 3H); ¹³C NMR (600 MHz, D₂O, pD 8.0) δ 183.74, 183.39, 166.57, 165.53, 157.74, 135.96, 132.44, 126.76, 125.18, 124.73, 122.32, 119.95, 119.34, 118.01, 46.56, 37.13; ESI-TOF-MS (m/z): [M+H]⁺ Calculated for C₁₈H₁₅N₂O₆ 355.0737, obtained 355.0768.

The modelling reactions were performed in the following photo-reactor (HepatoChem Inc.) fitted with a water bath attached to a recirculating pump to maintain a constant temperature of the water bath. The reaction can be performed using an air or oxygen balloon. PhotoCLIC protein labelling can also be performed in this photoreactor (placing the PhotoCLIC labelling cocktail in a 2 mL Eppendorf or in 2 mL glass vial) without compromising the efficacy.

Synthesis of N-succinimidyl 4-[(tert-Butyloxycarbonyl)amino]-phenylacetate 11

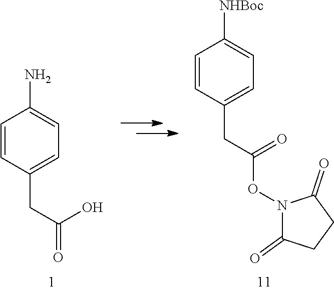 [SMILES: Nc1ccc(CC(=O)O)cc1 ; CC(C)(C)OC(=O)Nc1ccc(CC(=O)ON2C(=O)CCC2=O)cc1]

N-succinimidyl 4-[(tert-Butyloxycarbonyl)amino]-phenylacetate (11) was prepared from 4-aminophenylacetic acid 1 (TCI America) following a literature procedure.^{[4]}
Synthesis of alkyne-PEG3-aniline

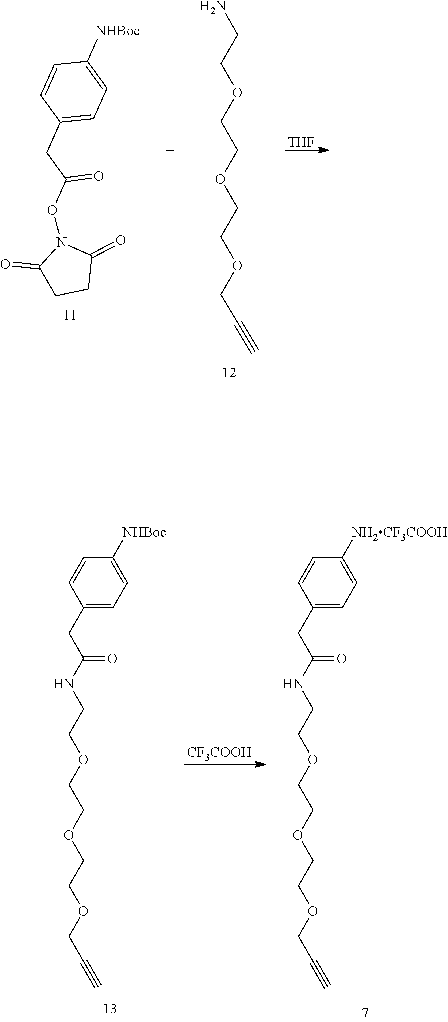

A mixture of alkyne-peg3-amine 12 (375 mg, 2.0 mmol; Lumiprobe), N-succinimidyl ester 11 (700 mg, 2.01 mmol) in anhydrous THF (1 mL) was heated and stirred at 60° C. for 8 h to have full conversion of the starting N-succinimidyl ester (judged by LC-MS). The solvent was evaporated off under reduced pressure and the residue was purified by silica gel (100-200 mesh) column chromatography (gradient elution: 50% EtOAc in hexanes to EtOAc to 2% MeOH in EtOAc) to afford the product. Silica gel was removed from the product by dissolving the product in warm chloroform and filtering. Concentrating the filtrate provided the intermediate 13 as a yellow oil (722 mg, 1.72 mmol; 86% yield). Rf: 0.40 (1% MeOH in EtOAc); ¹H-NMR (500 MHz, CDCl₃): δ 7.33 (d, J=10 Hz; 2H), 7.16 (d, J=10; 2H), 6.76 (s; 1H), 6.04 (s; 1H), 4.17-4.18 (m; 2H), 3.61-3.68 (m; 4H), 3.52-3.56 (m; 4H), 3.47-3.49 (m; 4H), 3.37-3.40 (m; 2H), 2.42-2.43 (m; 1H), 1.50 (s; 9H); ¹³C-NMR (125 MHz, CDCl₃): δ 171.55, 153.08, 137.88, 130.18, 129.74, 119.24, 80.75, 79.85, 74.97, 70.87, 70.77, 70.60, 70.50, 69.99, 60.66, 58.66, 43.29, 39.84, 28.62.

To the intermediate (13) was dissolved in 5 mL CH₂Cl₂ and anhydrous trifluoroacetic acid (5 mL) was added dropwise at 4° C. and the resulting mixture was stirred at 4° C. for 5 h to have complete removal of Boc protection (judged by LC-MS). The mixture was concentrating under reduced pressure. To the resulting oil CH₂Cl₂ (˜10 mL) was added and concentrated to dryness. The process was repeated four times to get rid of dissolved trifluoroacetic acid. The expected product (7) was obtained as a yellow oil (545 mg, 1.7 mmol, quantitative yield). ¹H-NMR (500 MHz, CD₃OD): δ 7.22 (d, J=10 Hz; 2H), 7.10 (d, J=10; 2H), 5.24 (s; 1H), 3.94 (d, J=5; 2H), 3.35-3.44 (m; 9H), 3.29-3.31 (m; 2H), 3.07 (s; 1H), 2.60 (d, J=5; 1H); ¹³C-NMR (125 MHz, CD₃OD): δ 173.58, 138.74, 132.23, 130.98, 124.42, 80.82, 76.28, 71.83, 71.61, 71.50, 70.70, 70.36, 59.33, 43.23, 40.90. ESI-TOF-MS (m/z): [M+H]⁺ Calculated for C₁₇H₂₅N₂O₄ 321.1814, obtained 321.1799.

Synthesis of Biotin-PEG3-aniline

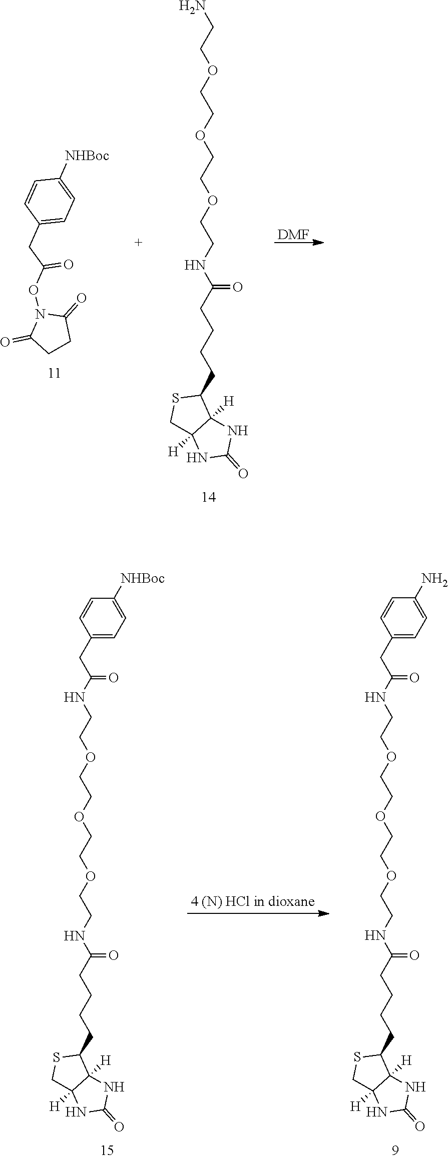

A mixture of Biotin-PEG3-amine 14 (420 mg, 1.0 mmol; BroadPharm), N-succinimidyl ester 11 (419 mg, 1.2 mmol) in anhydrous DMF (1 mL) was heated and stirred at 50° C. for 12 h. The solvent was evaporated off under reduced pressure and the residue was purified by silica gel (100-200 mesh) column chromatography (gradient elution: EtOAc to 2% MeOH in EtOAc to 10% MeOH in EtOAc and then to 25% MeOH in EtOAc) to afford the product. Silica gel was removed from the product by dissolving the product in warm chloroform and filtering. The intermediate 15 was obtained as a yellow oil (508 mg, 0.78 mmol; 78% yield). Rf: 0.60 (50% MeOH in EtOAc); ¹H-NMR (500 MHz, CDCl₃): 7.32-7.33 (m; 2H), 715-7.16 (m; 2H), 6.76 (s; 1H), 6.37-6.43 (m; 2H), 6.84 (s; 1H), 4.45-4.47 (m; 1H), 4.25-4.27 (m; 1H), 4.08-4.12 (m; 1H), 3.48-3.57 (m; 12H), 3.35-3.41 (m; 3H), 3.08-3.12 (m; 1H), 2.84-2.88 (m; 1H), 2.65-2.72 (m; 3H), 2.16-2.19 (m; 2H), 1.58-1.68 (m; 4H), 1.49 (s; 9H), 1.37-1.41 (m; 2H), 1.22-1.25 (m; 3H); ¹³C-NMR (125 MHz, CDCl₃): δ 173.91, 171.98, 164.69, 153.34, 137.96, 130.16, 129.80, 119.34, 81.42, 70.66, 70.63, 70.45, 70.33, 70.14, 70.01, 62.24, 60.69, 55.83, 43.15, 40.73, 39.69, 39.47, 36.11, 28.67, 25.79, 25.73, 21.33. ESI-TOF-MS (m/z): [M+H]⁺ Calculated for C₃₁H₅₀N₅O₈S 652.3374, obtained 652.3164.

To the intermediate (15) a solution of 4 (N) HCl in 1,4-dioxane (10 mL) was added dropwise at 4° C. and the resulting mixture was allowed to come to the room temperature while stirred. A solid precipitate started forming. After 4 h stirring at the room temperature the mixture was concentrating under reduced pressure. To the resulting oil chloroform (˜15 mL) was added and concentrated to dryness to get rid of dissolved HCl. The oil which was triturated with diethylether (˜20 mL) and dried thoroughly. Resulting solid residue was dissolved in ˜10 mL water and lyophilized to get the expected product (9) as a yellow solid (410 mg, 0.74 mmol, 95% yield). ¹H-NMR (600 MHz, D₂O): δ 7.38 (d, J=12 Hz; 2H), 7.33 (d, J=12; 2H), 4.49-4.52 (m; 1H), 4.30-4.32 (m; 1H), 3.74-3.76 (m; 1H), 3.65-3.68 (m; 1H), 3.59-3.60 (m; 8H), 3.53-3.56 (m; 4H), 3.29-3.34 (m; 4H), 3.20-3.23 (m; 1H), 2.88-2.91 (m; 1H), 2.67-2.71 (m; 1H), 2.16-2.19 (m; 2H), 1.43-1.65 (m; 4H), 1.27-1.33 (m; 2H); ¹³C-NMR (150 MHz, D₂O): δ 176.72, 173.75, 165.08, 136.27, 130.66, 128.62, 123.22, 71.49, 70.63, 69.56, 69.34, 68.75, 68.67, 62.00, 60.19, 55.25, 43.20, 41.62, 39.62, 39.07, 38.86, 35.33, 27.82, 27.60, 25.06. ESI-TOF-MS (m/z): [M+H]⁺ Calculated for C₂₆H₄₂N₅O₆S 552.2361, obtained 552.2388.

Synthesis of the carboxyfluorescein-aniline (10):

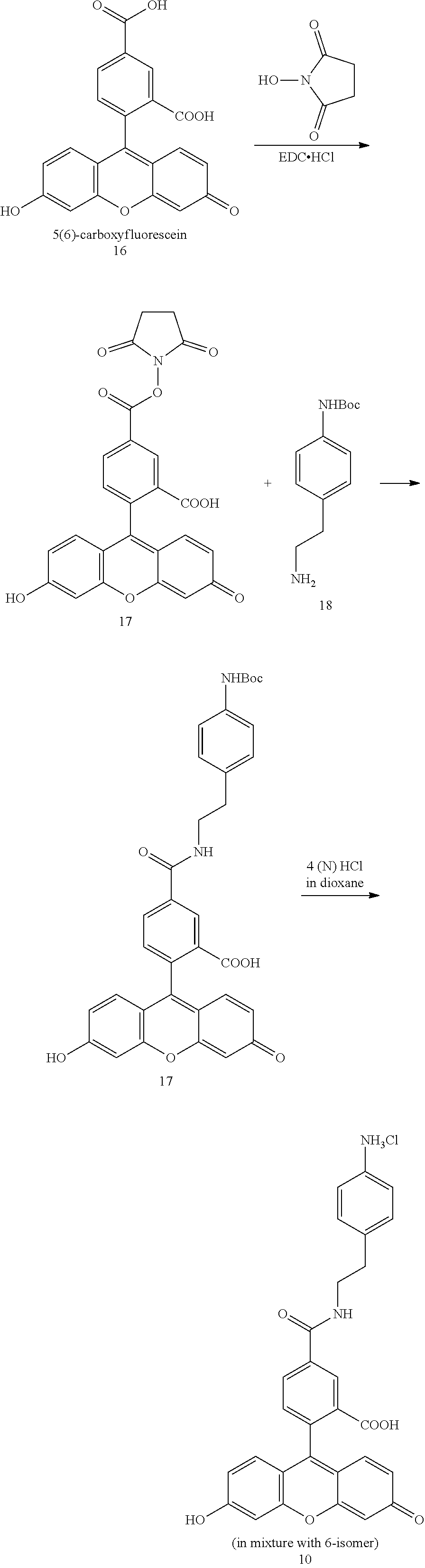

To a solution of 5(6)-carboxyfluorescein 16 (130 mg, 0.35 mmol; mixture of 5- and 6-isomer; Cayman Chemical) in anhydrous DMF (0.5 mL) was added 1-[3-(dimethylamino)propyl]-3-ethylcarbodiimide hydrochloride (EDC·HCl, 76 mg, 0.4 mmol; Chem Impex Int.) followed by the addition of N-hydroxysuccinimide (52 mg, 0.45 mmol) and the resulting mixture was stirred at the room temperature for 16 h. Solvent was evaporated off under reduced pressure and the residue was dissolved in a mixture of THF/EtOAc (50 mL, 1:9) and washed with water (4×15 mL). Combined organic layer was dried over anhydrous Na₂SO₄ and concentrated under reduced pressure to get the N-succinimidyl ester of 5(6)-carboxyfluorescein 17 (126 mg, 0.26 mmol, 74% yield). The purity of the product was confirmed by HPLC-MS analysis. ESI-TOF-MS (m/z): [M+H]⁺ Calculated for C₂₅H₁₆NO₉ 474.0825, obtained 474.0620. The intermediate was used directly in the next step without further purification.

N-Boc-protected phenethylamine (18) was prepared following a reported protocol from 4-aminophenethylamine (Combi-Blocks).^{[68]}

To a solution of N-succinimidyl ester of 5(6)-carboxyfluorescein 17 (120 mg, 0.25 mmol) in THF (5 mL) was added N-Boc-protected phenethylamine 18 (70 mg, 0.3 mmol) and the resulting mixture was stirred at the room temperature for 20 h to have the complete conversion (LC-MS). The solvent was evaporated off under reduced pressure and the residue was partitioned between a biphasic mixture of water (25 mL) and organic solvents (EtOAc/THF 9:1, 40 mL). Organic layer was separated and aqueous layer was extracted with the EtOAc/THF mixture (3×15 mL). Combined organic layer was dried over anhydrous Na₂SO₄ and concentrated under reduced pressure to get the expected product 19 as a red semi-solid (118 mg, 0.2 mmol, 80% yield). The purity of the product was confirmed by HPLC-MS analysis. ESI-TOF-MS (m/z): [M+H]⁺ Calculated for C₃₄H₃₁N₂O₈ 595.2080, obtained 595.1850. The intermediate was used directly in the next step without further purification.

Anhydrous 4 (N) HCl solution in 1,4-dioxane (5 mL) was added to the intermediate 19 under an inert atmosphere. The resulting mixture was stirred for 4 h at the room temperature. LC-MS analysis of the crude mixture confirmed completion of the removal of the Boc protection. Solvent was evaporated off under reduced pressure to get an oil that was repeatedly diluted with 15 mL chloroform and dried under reduced pressure to get rid of dissolved hydrochloric acid. The expected product 10 was obtained as an orange-yellow solid (105 mg, quantitative yield). The purity of the product was confirmed by HPLC-MS analysis. ESI-TOF-MS (m/z): [M+H]⁺ Calculated for C₂₉H₂₃N₂O₆ 495.1556, obtained 495.1436.

### Example 9: Screening

Screening to find conjugating partners for 5HTP under photoredox conditions using sfGFP-151-5HTP as the model. An initial screen of potential reaction partners was performed using LC-MS. We choose to screen following organic dyes traditionally used as histological stains as photocatalysts as they are soluble in an aqueous buffer and has absorption in the visible region of the spectrum: methylene blue, rose bengal, eyosin-Y, crystal violet, malachite green, bromophenol blue and commonly used organic photoredox catalysts [e.g. Ru(bpy)₃²⁺]. Hepes was chosen as the buffer of choice for initial screening for its known (Analytical Biochemistry 1988, 173 (1), p. 111-5) ability to suppress protein overoxidation by quenching unproductive reactive oxygen species. The absorption spectra of a panel of organic dyes (10 μM) were recorded in HEPES buffer (100 mM) to find suitable wavelength for promoting a reaction with a particular dye/photocatalyst. Among the dyes/photocatalysts it was observed that methylene blue has the most red shifted absorption maxima (λₘₐₓ: 664 nm) and hence was chosen as the initial catalyst (10 mol % of the starting protein) in the presence of red LED (650 nm) to screen different coupling partners with sfGFP-151-5HTP.

TABLE 1

| | | Maxium |
|---|---|---|
| | Organic | absorption |
| Entry | dye/photocatalyst | λₘₐₓ |
| 1 | eosin-Y | 516 nm |
| 2 | xylene cyanol FF | 614 nm |
| 3 | malachite green | 618 nm |
| 4 | crystal violet | 590 nm |
| 5 | bromothymol blue | 616 nm |
| 6 | blue dextran 2000 | 624 nm |
| 7 | bromophenol blue | 592 nm |
| 8 | rose bengal | 548 nm |
| 9 | methylene blue | 664 nm |
| 10 | riboflavin Na-phosphate | 448 nm |
| 11 | [Ru(bpy)₃]²⁺ 2Cl | 550 nm |

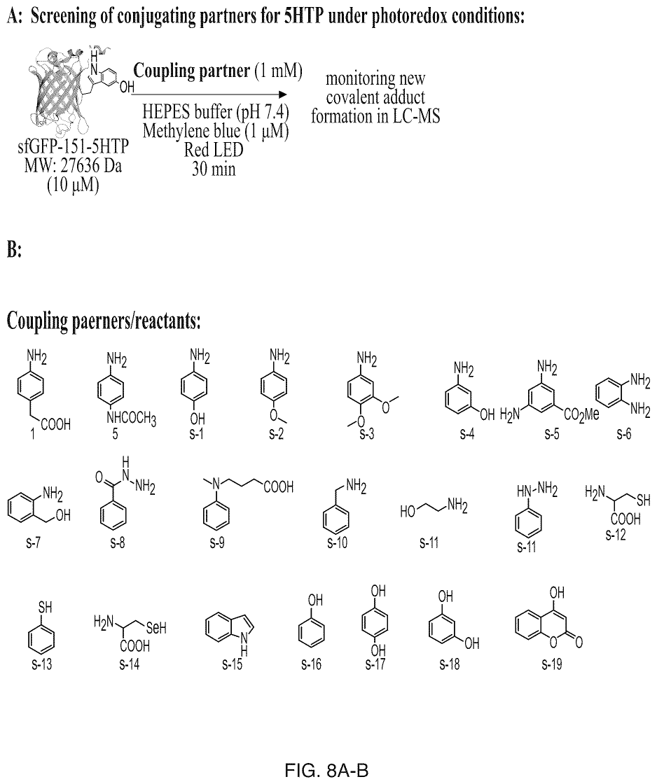

Structures of coupling partners/reactants are shown in FIGS. 8A-B. Results are shown in Table 2, below.

TABLE 2

| Entry | Protein | Reactant | Comment/Observation |
|---|---|---|---|
| 1 | sfGFP-151-5HTP | 1 | A new covalent adduct with 27799 |
| | | | Da mass (27636 + 151 + 12) |
| 2 | WT-sfGFP | 1 | No new covalent adduct |
| 5 | sfGFP-151-5HTP | 5 | A new covalent adduct with 27798 |
| | | | Da mass (27636 + 150 + 12) |
| 6 | WT-sfGFP | 5 | No new covalent adduct |
| 3 | sfGFP-151-5HTP | s-4 | A new covalent adduct with 27757 |
| | | | Da mass (27636 + 109 + 12) |
| 4 | WT-sfGFP | s-4 | No new covalent adduct |
| 7 | sfGFP-151-5HTP | s-1 to s-19 | No new covalent adduct |
| | | except s-4 | |
| 8 | WT-sfGFP | s-1 to s-19 | ND |
| | | except s-4 | |

Optimization of photoCLIC conditions for labeling of anilines with 5HTP was performed using sfGFP-151-5HTP at 10 μM with a photocatalyst, varying buffers, aeration, temperature and time. Results are shown in Table 3 below.

TABLE 3

| | Light | | Buffer | Conc. of | Temperature | Time | |
|---|---|---|---|---|---|---|---|
| Entry | source | Photocatalyst^{a} | (pH) | 1 (mM) | (° C.) | (min) | Conversion^{b} |
| 1 | Red LED | Methylene | HEPES | 1 | 4 | 30 | ~80%^{c} |
| | (650 nm) | blue | (pH 7.4) | | | | |
| 2 | Green | Methylene | HEPES | 1 | 4 | 30 | ~80%^{c} |
| | LED | blue | (pH 7.4) | | | | |
| | (520 nm) | | | | | | |
| 3 | Blue LED | Methylene | HEPES | 1 | 4 | 30 | ~80%^{c} |
| | (455 nm) | blue | (pH 7.4) | | | | |
| 4 | Blue LED | Methylene | HEPES | 2 | 4 | 30 | ~90%^{d} |
| | (455 nm) | blue | (pH 7.4) | | | | |
| 5 | Blue LED | Rose Bengal | HEPES | 2 | 4 | 30 | ~75%^{c} |
| | (455 nm) | | (pH 7.4) | | | | |
| 6 | Blue LED | Eosin-Y | HEPES | 2 | 4 | 30 | ~70%^{c} |
| | (455 nm) | | (pH 7.4) | | | | |
| 7 | Blue LED | Riboflavin Na- | HEPES | 2 | 4 | 30 | ~70%^{c} |
| | (455 nm) | phosphate | (pH 7.4) | | | | |
| 8 | Blue LED | Xylene cyanol | HEPES | 2 | 4 | 30 | ~20%^{c} |
| | (455 nm) | FF | (pH 7.4) | | | | |
| 9 | Blue LED | [Ru(bpy)₃]²⁺ | HEPES | 2 | 4 | 30 | ~20%^{c} |
| | (455 nm) | 2CI | (pH 7.4) | | | | |
| 10 | Blue LED | Malachite | HEPES | 2 | 4 | 30 | ~15%^{c} |
| | (455 nm) | green | (pH 7.4) | | | | |
| 11 | Blue LED | Crystal violet | HEPES | 2 | 4 | 30 | ~20%^{c} |
| | (455 nm) | | (pH 7.4) | | | | |
| 12 | Blue LED | Bromothymol | HEPES | 2 | 4 | 30 | ~20%^{c} |
| | (455 nm) | blue | (pH 7.4) | | | | |
| 13 | Blue LED | Blue dextran | HEPES | 2 | 4 | 30 | ~20%^{c} |
| | (455 nm) | 2000 | (pH 7.4) | | | | |
| 14 | Blue LED | Bromothymol | HEPES | 2 | 4 | 30 | ~20%^{c} |
| | (455 nm) | blue | (pH 7.4) | | | | |
| 15 | Blue LED | Methylene | HEPES | 4 | 4 | 30 | ~94% |
| | (455 nm) | blue | (pH 7.4) | | | | |
| 16 | Blue LED | Methylene | HEPES | 4 | 4 | 35 | ~95% |
| | (455 nm) | blue | (pH 7.4) | | | | |
| 17 | Blue LED | Methylene | HEPES | 4 | 4 | 35 | >95% |
| | (455 nm) | blue | (pH 8.0) | | | | |
| 18 | Blue LED | Methylene | Phosphate | 4 | 4 | 35 | ~90% |
| | (455 nm) | blue | buffer | | | | |
| | | | (pH 7.4) | | | | |
| 19 | Blue LED | Methylene | Phosphate | 4 | 4 | 35 | ~95% |
| | (455 nm) | blue | buffer | | | | |
| | | | (pH 8.0) | | | | |
| 20 | Blue LED | Methylene | HEPES | 4 | RT | 35 | >95% |
| | (455 nm) | blue | (pH 8.0) | | | | |
| 21 | Blue LED | Methylene | HEPES | 4 | 4 | 35 | 0% |
| | (455 nm) | blue | (pH 8.0) | | | | |
| | | | deoxygeneted | | | | |
| 22 | Blue LED | Methylene | HEPES | 4 | 4 | 35 | ~90% |
| | (455 nm) | blue (0.5 μM) | (pH 8.0) | | | | |
| 23 | Blue LED | Methylene | HEPES | 4 | 4 | 35 | >95%^{c} |
| | (455 nm) | blue (2 μM) | (pH 8.0) | | | | |
| 24 | No | Methylene | HEPES | 4 | 4 | 35 | 0% |
| | irradiation | blue | (pH 8.0) | | | | |

^{a}unless mentioned otherwise 1 μM photocatalyst was used.
^{b}conversion to product (%) assessed by relative intensities of intact protein mass (ESI) recorded in an ESI-TOF LC/MS system.
^{c}product shows off-target oxidation (ESI-MS).
^{d}marginally reduced off-target protein oxidation. RT: room temperature (22° C.)

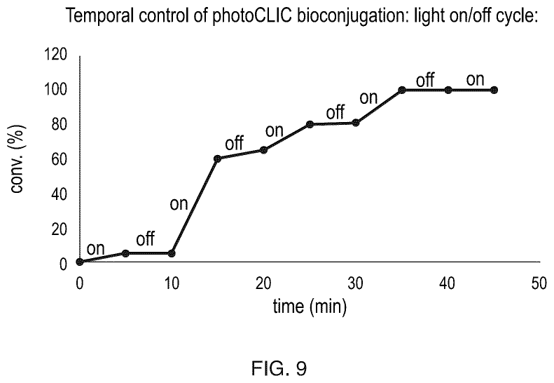

Time course of the PhotoCLIC labeling of sfGFP-151-5HTP with reactant 1(sfGFP-151-5HTP) using whole-protein MS analysis and corresponding raw mass spec data (data not shown) Demonstration of temporal control of the PhotoCLIC labeling through following light ON/OFF cycle: 5 min ON then 5 min OFF, 5 min ON then 5 min OFF, 5 min ON 5 min OFF, 5 min ON, 5 min OFF, 5 min ON as shown in FIG. 9.

### Example 10: Analysis of Wild-Type and Mutated Anti-HER2-Nanobody, Myoglobin and Nonoluciferase

MS analysis of wild-type and 5HTP mutated anti-Her2-nanobody, myoglobin and nanoluciferase; biotin labeling of these three recombinant proteins using PhotoCLIC.

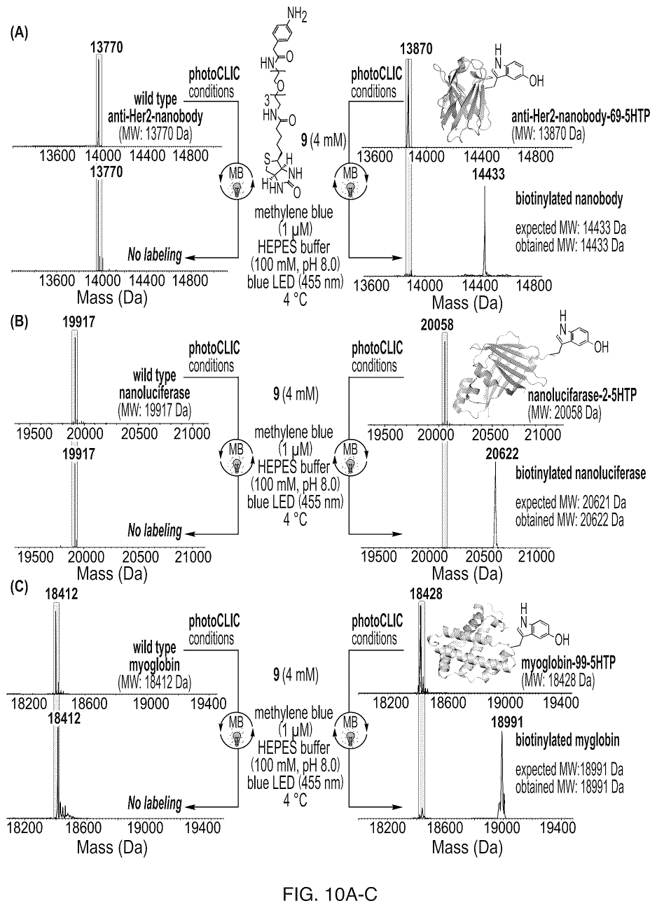

Corresponding wild type proteins showed no labeling under otherwise identical conditions as shown in FIG. 10 A-C. Amino acid sequences are shown in the sequence section. Raw MS data is not shown.

### Example 11: Trustuzumab-HC-198-5HTP

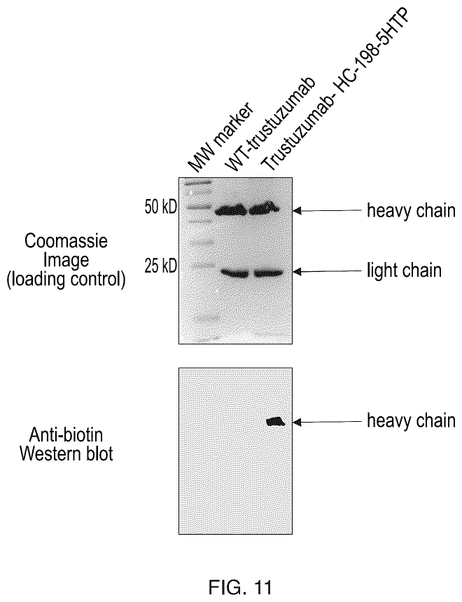

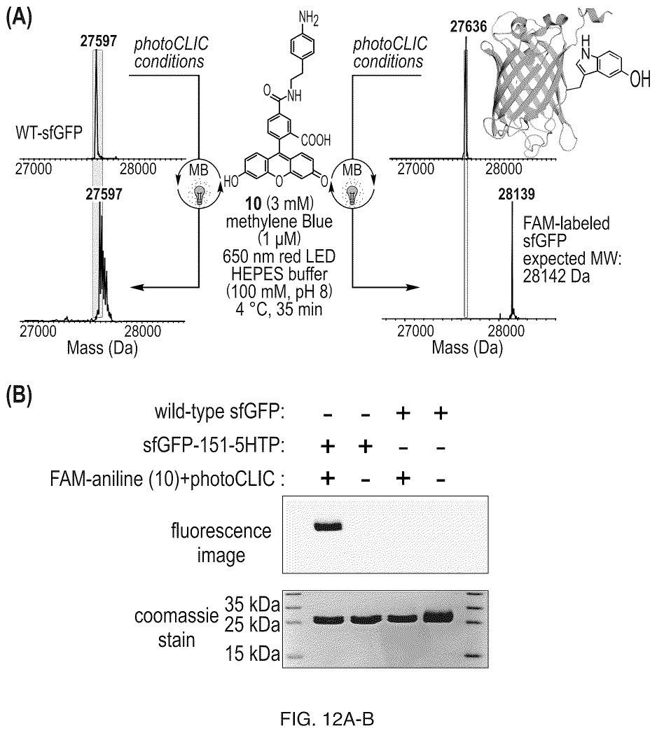

Using an engineered EcTrpRS/tRNA pair, 5HTP was site-specifically incorporated into the 198 site of the heavy chain of Trastuzumab (MS data not shown). PhotoCLIC labeling of Trustuzumab-HC-198-5HTP was demonstrated with biotin-PEG3-aniline 9 (MS data not shown). Selective biotinylation of Trustuzumab HC-198-5HTP with 9 under photoCLIC conditions, revealed by anti-biotin Western blot (using a streptavidin-HRP probe). Both WT-Trustuzumab and Trustuzumab HC-198-5HTP were subjected to biotinylation under photoCLIC conditions, desalted and resolved in SDS-PAGE (reductive denaturing) gel followed by Western blot as shown in FIG. 11 A-B.

### Example 12: Plasmid Maps

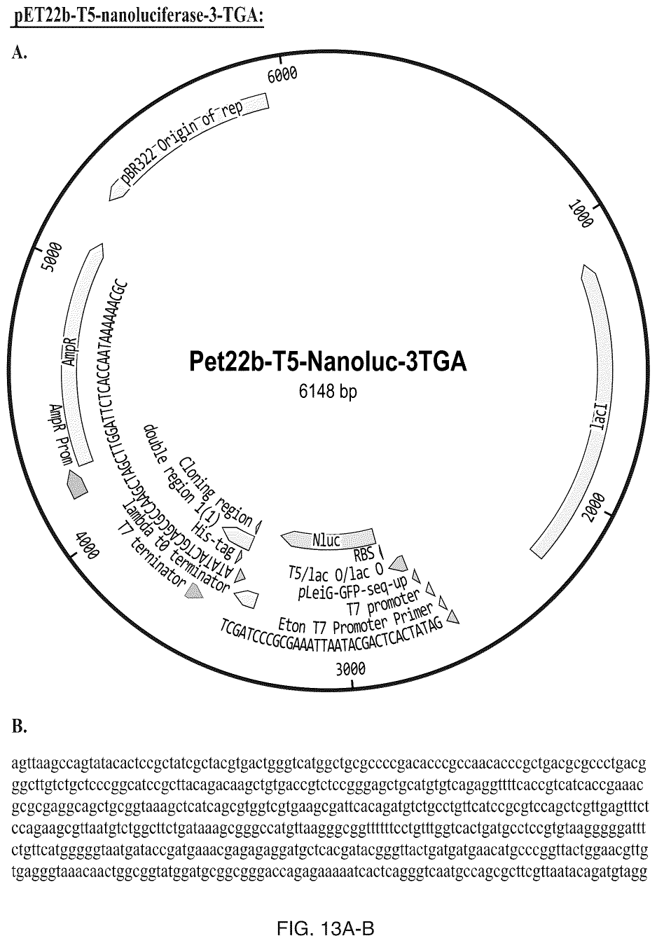

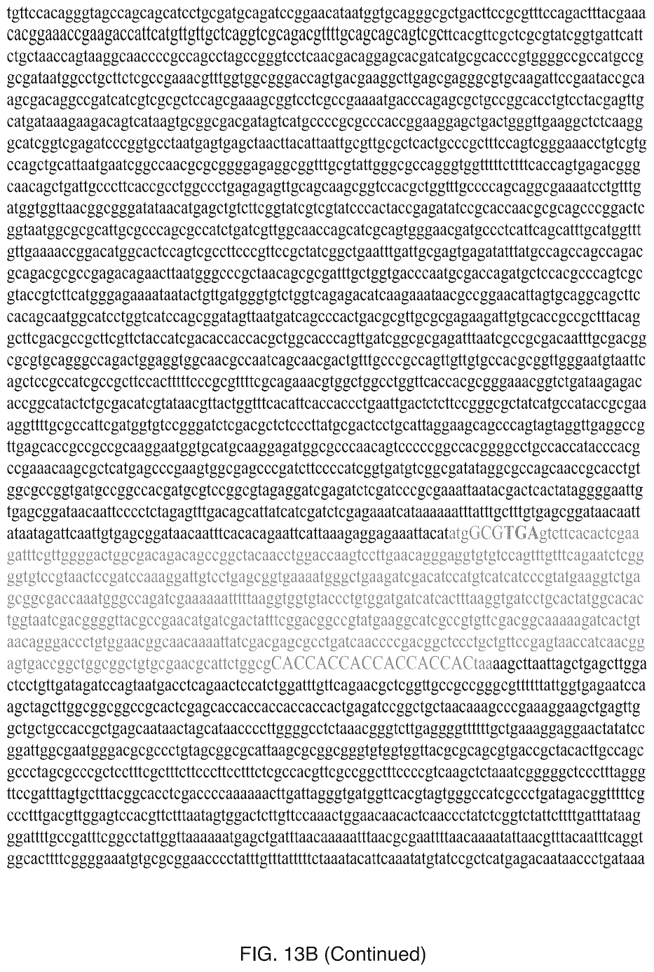

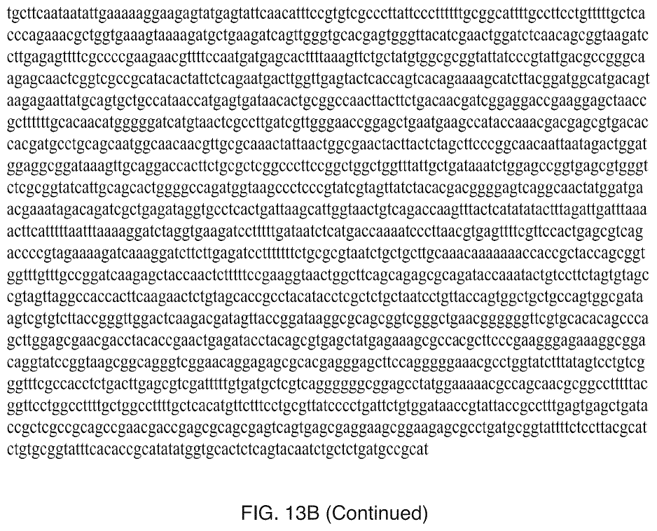

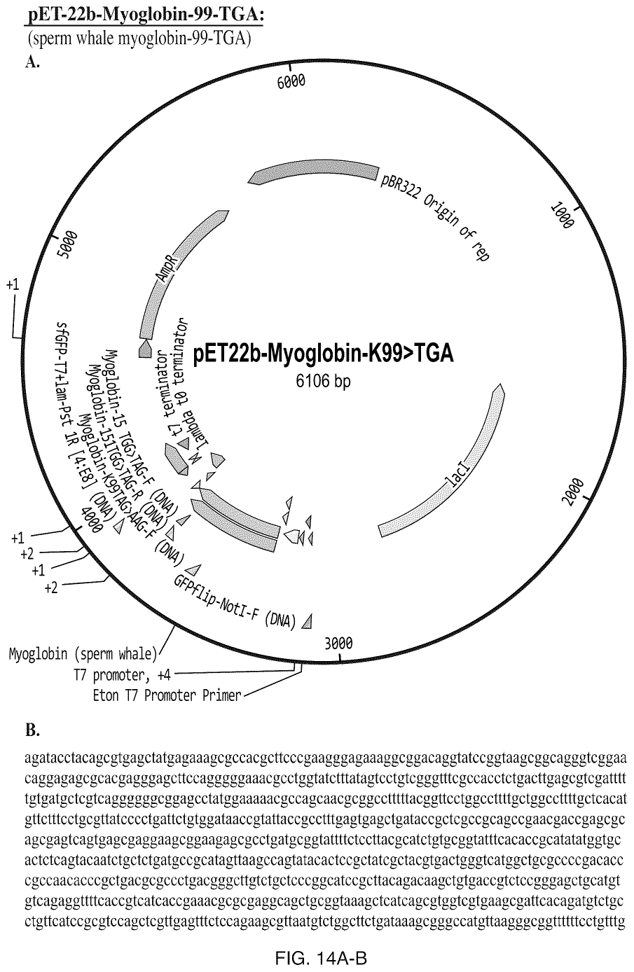

Plasmid maps and sequences of pET22b-T5-nonoluciferase-3-TGA (FIG. 13 A-B) (SEQ ID NO: 16)

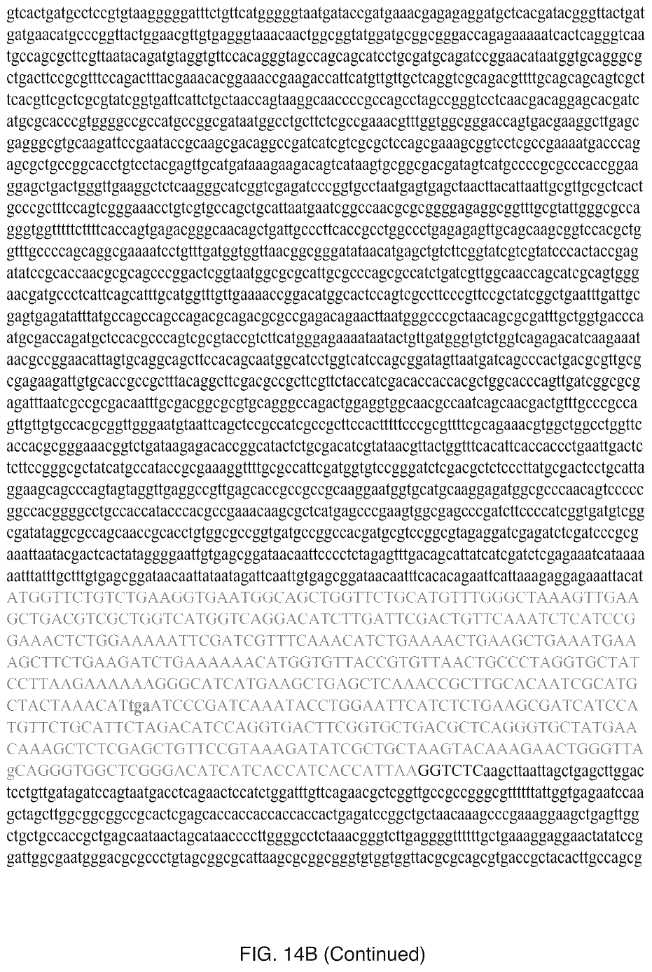

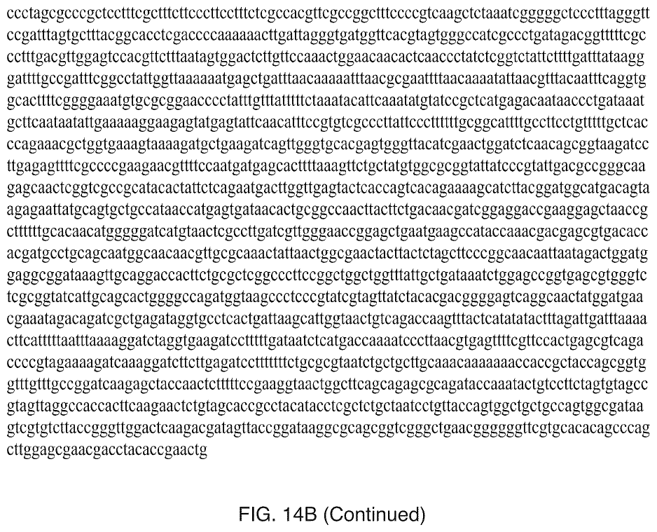

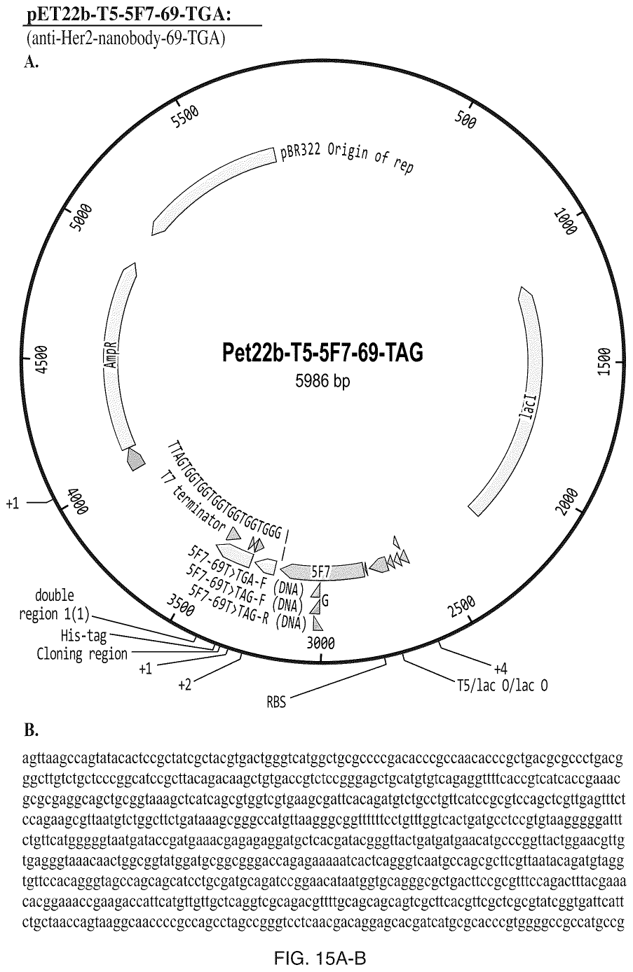

pET-22b-Myoglobin-99-TGA (sperm whale myoglobin-99-TGA) (FIG. 14A-B) (SEQ ID NO:17)

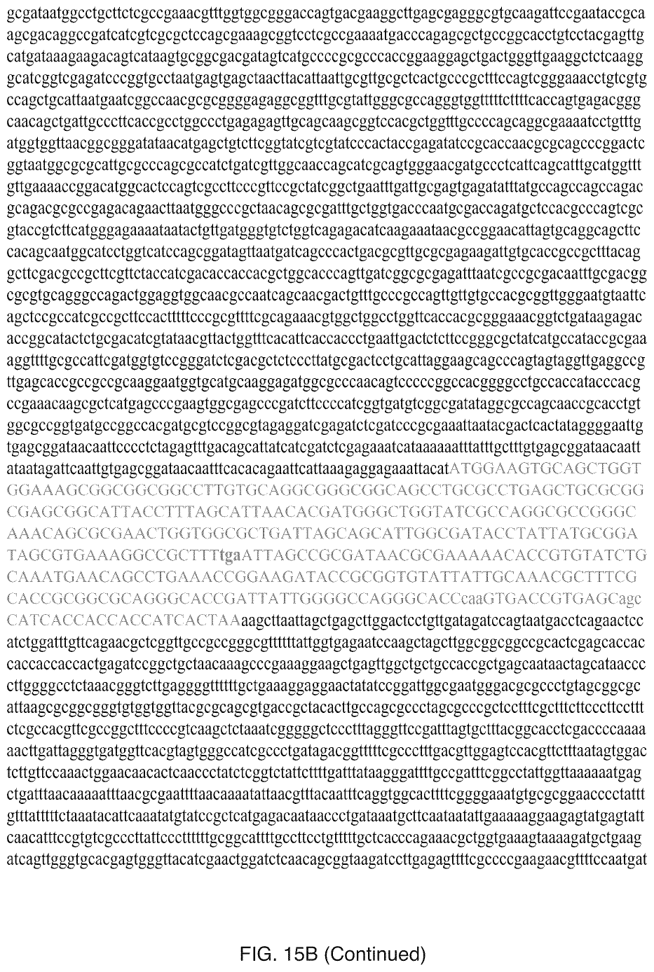

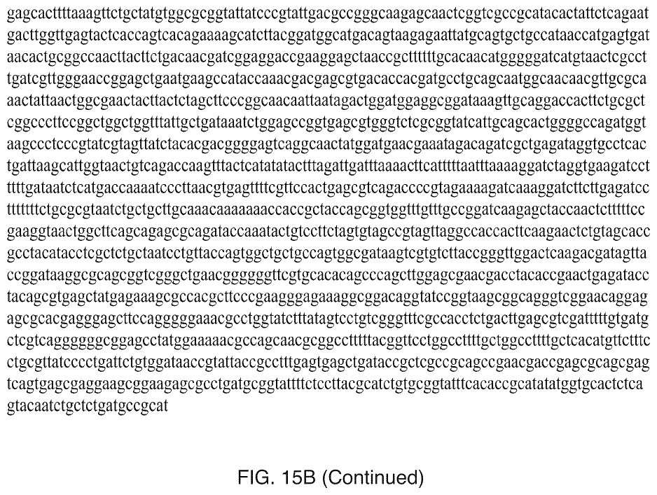

pET22b-T5-SF7-69-TGA (anti-Her2-nanobody-69-TGA) (FIG. 15 A-B)(SEQ ID NO:18)

### Example 13: Sequences

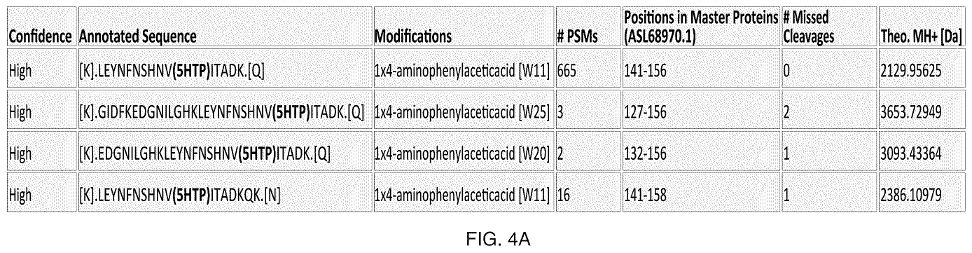

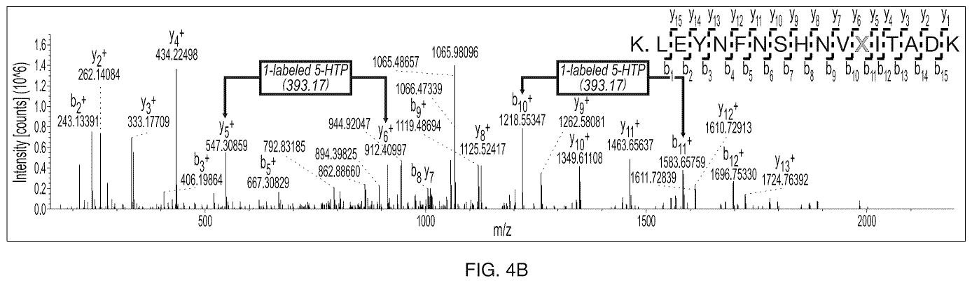

Amino acid sequence of sfGFP-151-5HTP and wild type sfGFP:
sfGFP-151-5HTP (SEQ ID NO: 1):
MSKGEELFTGVVPILVELDGDVNGHKFSVRGEGEGDATNGKLTLKFICTTGKL
PVPWPTLVTTLTYGVQCFSRYPDHMKRHDFFKSAMPEGYVQERTISFKDDGT
YKTRAEVKFEGDTLVNRIELKGIDFKEDGNILGHKLEYNFNSHNV(5HTP)ITAD
KQKNGIKANFKIRHNVEDGSVQLADHYQQNTPIGDGPVLLPDNHYLSTQSVL
SKDPNEKRDHMVLLEFVTAAGITHGMDELYKGSHHHHHH
WT-sfGFP (SEQ ID NO: 2):
MSKGEELFTGVVPILVELDGDVNGHKFSVRGEGEGDATNGKLTLKFICTTGKL
PVPWPTLVTTLTYGVQCFSRYPDHMKRHDFFKSAMPEGYVQERTISFKDDGT
YKTRAEVKFEGDTLVNRIELKGIDFKEDGNILGHKLEYNFNSHNVY(151)ITAD
KQKNGIKANFKIRHNVEDGSVQLADHYQQNTPIGDGPVLLPDNHYLSTQSVL
SKDPNEKRDHMVLLEFVTAAGITHGMDELYKGSHHHHHH
5-HTP Peptide Fragments: (from FIG. 4A)
LEYNFNSHNV(5HTP)ITADK (SEQ ID NO: 3)
GIDFKEDGNILGHKLEYNFNSHNV(5HTP)ITADK (SEQ ID NO: 4)
EDGNILGHKLEYNFNSHNV(5HTP)ITADK (SEQ ID NO: 5)
LEYNFNSHNV(5HTP)ITADKQK (SEQ ID NO: 6)
Amino acid Sequence of anti-her2-nanobody-69-5HTP and wild type anti-her2-nanobody:
anti-her2-nanobody-69-5HTP (SEQ ID NO: 7):
MEVQLVESGGGLVQAGGSLRLSCAASGITFSINTMGWYRQAPGKQRELVALISSIG
DTYYADSVKGRFT(5HTP)SRDNAKNTVYLQMNSLKPEDTAVYYCKRFRTAAQGT
DYWGQGTQVTVSSHHHHHH
wild type anti-her2-nanobody (SEQ ID NO: 8):
MEVQLVESGGGLVQAGGSLRLSCAASGITFSINTMGWYRQAPGKQRELVALISSIG
DTYYADSVKGRFTI(69)SRDNAKNTVYLQMNSLKPEDTAVYYCKRFRTAAQGTD
YWGQGTQVTVSSHHHHHH
Amino acid Sequence of myoglobin-99-5HTP and wild type myoglobin:
myoglobin-99-5HTP SEQ ID NO: 9):
MVLSEGEWQLVLHVWAKVEADVAGHGQDILIRLFKSHPETLEKFDRFKHLKT
EAEMKASEDLKKHGVTVLTALGAILKKKGHHEAELKPLAQSHATKH(5HTP)I
PIKYLEFISEAIIHVLHSRHPGDFGADAQGAMNKALELFRKDIAAKYKEL
GYQGGSGHHHHHH
wild type myoglobin (SEQ ID NO: 10):
MVLSEGEWQLVLHVWAKVEADVAGHGQDILIRLFKSHPETLEKFDRFKHLKTEA
EMKASEDLKKHGVTVLTALGAILKKKGHHEAELKPLAQSHATKHW(99)IPIKYLEF
ISEAIIHVLHSRHPGDFGADAQGAMNKALELFRKDIAAKYKELGYQGGSGHHH
HHH
Amino acid Sequence of Nanoluciferase-2-5HTP and wild type nanoluciferase:
Nanoluciferase-2-5HTP (SEQ ID NO: 11):
A(5HTP)VFTLEDFVGDWRQTAGYNLDQVLEQGGVSSLFQNLGVSVTPIQRIVLSGE
NGLKIDIHVIIPYEGLSGDQMGQIEKIFKVVYPVDDHHFKVILHYGTLVIDGVTPNM
IDYFGRPYEGIAVFDGKKITVTGTLWNGNKIIDERLINPDGSLLFRVTINGVTGWRL
CERILAHHHHHH
Wild-type-nanoluciferase (SEQ ID NO: 12):
MVFTLEDFVGDWRQTAGYNLDQVLEQGGVSSLFQNLGVSVTPIQRIVLSGENGLK
IDIHVIIPYEGLSGDQMGQIEKIFKVVYPVDDHHFKVILHYGTLVIDGVTPNMIDYF
GRPYEGIAVFDGKKITVTGTLWNGNKIIDERLINPDGSLLFRVTINGVTGWRLCERI
LAHHHHHH
Amino acid Sequence of Trastuzumab-HC-198-5HTP and wild type Trastuzumab:
light chain (SEQ ID NO: 13):
DIQMTQSPSSLSASVGDRVTITCRASQDVNTAVAWYQQKPGKAPKLLIYSASFLYS
GVPSRFSGSRSGTDFTLTISSLQPEDFATYYCQQHYTTPPTFGQGTKVEIKRTVAAP
SVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
heavy chain-wild type (SEQ ID NO: 14):
EVQLVESGGGLVQPGGSLRLSCAASGFNIKDTYIHWVRQAPGKGLEWVARIY
PTNGYTRYADSVKGRFTISADTSKNTAYLQMNSLRAEDTAVYYCSRWGGDG
FYAMDYWGQGTLVTVSSASTKGPSVFPLAPSSKSTSGGTAALGCLVKDYFPE
PVTVSWNSGALTSGVHTFPAVLQSSGLYSLSSVVTVPSSSLGT(198)QTYICNV
NHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVD
KSRWQQGNVFSCSVMHEALHNHYTQKSLSLSPGS
heavy chain with 198 site mutated to 5HTP (SEQ ID NO: 15):
EVQLVESGGGLVQPGGSLRLSCAASGFNIKDTYIHWVRQAPGKGLEWVARIY
PTNGYTRYADSVKGRFTISADTSKNTAYLQMNSLRAEDTAVYYCSRWGGDG
FYAMDYWGQGTLVTVSSASTKGPSVFPLAPSSKSTSGGTAALGCLVKDYFPE
PVTVSWNSGALTSGVHTFPAVLQSSGLYSLSSVVTVPSSSLG(5HTP)QTYICNV
NHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVD
KSRWQQGNVFSCSVMHEALHNHYTQKSLSLSPGS

### REFERENCES

The following references are incorporated herein in their entirety:
1. Scinto, S. L.; Bilodeau, D. A.; Hincapie, R.; Lee, W.; Nguyen, S. S.; Xu, M.; am Ende, C. W.; Finn, M. G.; Lang, K.; Lin, Q.; Pezacki, J. P.; Prescher, J. A.; Robillard, M. S.; Fox, J. M. Bioorthogonal Chemistry. Nat. Rev. Methods Prim. 2021, 1 (1), 30. https://doi.org/10.1038/s43586-021-00028-z.
2. Hoyt, E. A.; Cal, P. M. S. D.; Oliveira, B. L.; Bernardes, G. J. L. Contemporary Approaches to Site-Selective Protein Modification. Nat. Rev. Chem. 2019, 3 (3), 147-171. https://doi.org/10.1038/s41570-019-0079-1.
3. Tamura, T.; Hamachi, I. Chemistry for Covalent Modification of Endogenous/Native Proteins: From Test Tubes to Complex Biological Systems. J Am. Chem. Soc. 2019. https://doi.org/10.1021/jacs.8b11747.
4. Row, R. D.; Prescher, J. A. Constructing New Bioorthogonal Reagents and Reactions. Acc. Chem. Res. 2018, 51 (5), 1073-1081. https://doi.org/10.1021/acs.accounts.7b00606.
5. DeGruyter, J. N.; Malins, L. R.; Baran, P. S. Residue-Specific Peptide Modification: A Chemist's Guide. Biochemistry 2017, 56 (30), 3863-3873. https://doi.org/10.1021/acs.biochem.7b00536.
6. McKay, C. S.; Finn, M. G. Click Chemistry in Complex Mixtures: Bioorthogonal Bioconjugation. Chem. Biol. 2014, 21 (9), 1075-1101. https://doi.org/10.1016/j.chembiol.2014.09.002.
7. Stephanopoulos, N.; Francis, M. B. Choosing an Effective Protein Bioconjugation Strategy. Nat. Chem. Biol. 2011, 7 (12), 876-884. https://doi.org/10.1038/nchembio.720.
8. Sletten, E. M.; Bertozzi, C. R. Bioorthogonal Chemistry: Fishing for Selectivity in a Sea of Functionality. Angew. Chemie-Int. Ed. 2009, 48 (38), 6974-6998. https://doi.org/10.1002/anie.200900942.
9. Shadish, J. A.; DeForest, C. A. Site-Selective Protein Modification: From Functionalized Proteins to Functional Biomaterials. Matter 2020, 2 (1), 50-77. https://doi.org/10.1016/j.matt.2019.11.011.
10. Hu, Q. Y.; Berti, F.; Adamo, R. Towards the next Generation of Biomedicines by Site-Selective Conjugation. Chem. Soc. Rev. 2016, 45 (6), 1691-1719. https://doi.org/10.1039/c4cs00388h.
11. Koniev, O.; Wagner, A. Developments and Recent Advancements in the Field of Endogenous Amino Acid Selective Bond Forming Reactions for Bioconjugation. Chem. Soc. Rev. 2015, 44 (15), 5495-5551. https://doi.org/10.1039/c5cs00048c.
12. Diercks, C. S.; Dik, D. A.; Schultz, P. G. Adding New Chemistries to the Central Dogma of Molecular Biology. Chem 2021, 7 (11), 2883-2895. https://doi.org/10.1016/j.chempr.2021.09.014.
13. Manandhar, M.; Chun, E.; Romesberg, F. E. Genetic Code Expansion: Inception, Development, Commercialization. J. Am. Chem. Soc. 2021, 143 (13), 4859-4878. https://doi.org/10.1021/jacs.0c11938.
14. Dumas, A.; Lercher, L.; Spicer, C. D.; Davis, B. G. Designing Logical Codon Reassignment-Expanding the Chemistry in Biology. Chem. Sci. 2015, 6 (1), 50-69. https://doi.org/10.1039/c4sc01534g.
15. Lang, K.; Chin, J. W. Cellular Incorporation of Unnatural Amino Acids and Bioorthogonal Labeling of Proteins. Chem. Rev. 2014, 114 (9), 4764-4806. https://doi.org/10.1021/cr400355w.
16. Hu, Y.; Schomaker, J. M. Recent Developments and Strategies for Mutually Orthogonal Bioorthogonal Reactions. ChemBioChem 2021, 22 (23), 3254-3262. https://doi.org/10.1002/cbic.202100164.
17. Italia, J. S.; Addy, P. S.; Erickson, S. B.; Peeler, J. C.; Weerapana, E.; Chatterjee, A. Mutually Orthogonal Nonsense-Suppression Systems and Conjugation Chemistries for Precise Protein Labeling at up to Three Distinct Sites. J. Am. Chem. Soc. 2019, 141 (15), 6204-6212. https://doi.org/10.1021/jacs.8b12954.
18. Ficaretta, E. D.; Wrobel, C. J. J.; Roy, S. J. S.; Erickson, S. B.; Italia, J. S.; Chatterjee, A. A Robust Platform for Unnatural Amino Acid Mutagenesis in E. Coli Using the Bacterial Tryptophanyl-TRNA Synthetase/TRNA Pair. J. Mol. Biol. 2021, No. xxxx, 167304. https://doi.org/10.1016/j.jmb.2021.167304.
19. Italia, J. S.; Addy, P. S.; Wrobel, C. J. J.; Crawford, L. A.; Lajoie, M. J.; Zheng, Y.; Chatterjee, A. An Orthogonalized Platform for Genetic Code Expansion in Both Bacteria and Eukaryotes. Nat. Chem. Biol. 2017, 13 (4), 446-450. https://doi.org/10.1038/nchembio.2312.
20. Addy, P. S.; Zheng, Y.; Italia, J. S.; Chatterjee, A. A “Quenchergenic” Chemoselective Protein Labeling Strategy. ChemBioChem 2019, 20 (13), 1659-1663. https://doi.org/10.1002/cbic.201800817.
21. Addy, P. S.; Italia, J. S.; Chatterjee, A. An Oxidative Bioconjugation Strategy Targeted to a Genetically Encoded 5-Hydroxytryptophan. ChemBioChem 2018,
19 (13), 1375-1378. https://doi.org/10.1002/cbic.201800111.
22. Addy, P. S.; Erickson, S. B.; Italia, J. S.; Chatterjee, A. A Chemoselective Rapid Azo-Coupling Reaction (CRACR) for Unclickable Bioconjugation. J. Am. Chem. Soc. 2017, 139 (34), 11670-11673. https://doi.org/10.1021/jacs.7b05125.
23. Chen, Y.; Tang, J.; Wang, L.; Tian, Z.; Cardenas, A.; Fang, X.; Chatterjee, A.; Xiao, H. Creation of Bacterial Cells with 5-Hydroxytryptophan as a 21st Amino Acid Building Block. Chem 2020, 6 (10), 2717-2727. https://doi.org/10.1016/j.chempr.2020.07.013.
24. Wang, H.; Liu, W.; Shi, F.; Huang, L.; Lian, J.; Qu, L.; Cai, J.; Xu, Z. Metabolic Pathway Engineering for High-Level Production of 5-Hydroxytryptophan in Escherichia Coli. Metab. Eng. 2018, 48 (March), 279-287. https://doi.org/10.1016/j.ymben.2018.06.007.
25, Brauer, D. D.; Hartman, E. C.; Bader, D. L. V.; Merz, Z. N.; Tullman-Ercek, D.; Francis, M. B. Systematic Engineering of a Protein Nanocage for High-Yield, Site-Specific Modification. J. Am. Chem. Soc. 2019, 141 (9), 3875-3884. https://doi.org/10.1021/jacs.8b10734.
26. Kwant, R. L.; Jaffe, J.; Palmere, P. J.; Francis, M. B. Controlled Levels of Protein Modification through a Chromatography-Mediated Bioconjugation. Chem. Sci. 2015, 6 (4), 2596-2601. https://doi.org/10.1039/c4sc03790a.
27. Obermeyer, A. C.; Jarman, J. B.; Francis, M. B. N-Terminal Modification of Proteins with o-Aminophenols. J Am. Chem. Soc. 2014, 136 (27), 9572-9579. https://doi.org/10.1021/ja500728c.
28. Obermeyer, A. C.; Jarman, J. B.; Netirojjanakul, C.; El Muslemany, K.; Francis, M. B. Mild Bioconjugation through the Oxidative Coupling of Ortho-Aminophenols and Aromatic amines with Ferricyanide. Angew. Chemie-Int. Ed. 2014, 53 (4), 1057-1061. https://doi.org/10.1002/anie.201307386.
29. Isenegger, P. G.; Davis, B. G. Concepts of Catalysis in Site-Selective Protein Modifications. J Am. Chem. Soc. 2020, 141 (20), 8005-8013. https://doi.org/10.1021/jacs.8b13187.
30. Ryu, K. A.; Kaszuba, C. M.; Bissonnette, N. B.; Oslund, R. C.; Fadeyi, O. O. Interrogating Biological Systems Using Visible-Light-Powered Catalysis. Nat. Rev. Chem. 2021, 5 (5), 322-337. https://doi.org/10.1038/s41570-021-00265-6.
31. Tower, S. J.; Hetcher, W. J.; Myers, T. E.; Kuehl, N. J.; Taylor, M. T. Selective Modification of Tryptophan Residues in Peptides and Proteins Using a Biomimetic Electron Transfer Process. J. Am. Chem. Soc. 2020, 142 (20), 9112-9118. https://doi.org/10.1021/jacs.0c03039.
32. Chen, X.; Ye, F.; Luo, X.; Liu, X.; Zhao, J.; Wang, S.; Zhou, Q.; Chen, G.; Wang, P. Histidine-Specific Peptide Modification via Visible-Light-Promoted C-H Alkylation. J. Am. Chem. Soc. 2019, 141 (45), 18230-18237.
33. Garreau, M.; Le Vaillant, F.; Waser, J. C-Terminal Bioconjugation of Peptides through Photoredox Catalyzed Decarboxylative Alkynylation. Angew. Chemie-Int. Ed. 2019, 58 (24), 8182-8186. https://doi.org/10.1002/anie.201901922.
34. Bottecchia, C.; No81, T. Photocatalytic Modification of Amino Acids, Peptides, and Proteins. Chem.-A Eur. J. 2019, 25 (1), 26-42. https://doi.org/10.1002/chem.201803074.
34. Yu, Y.; Zhang, L. K.; Buevich, A. V.; Li, G.; Tang, H.; Vachal, P.; Colletti, S. L.; Shi, Z. C. Chemoselective Peptide Modification via Photocatalytic Tryptophan β-Position Conjugation. J. Am. Chem. Soc. 2018, 140 (22), 6797-6800. https://doi.org/10.1021/jacs.8b03973.
36. Li, B. X.; Kim, D. K.; Bloom, S.; Huang, R. Y. C.; Qiao, J. X.; Ewing, W. R.; Oblinsky, D. G.; Scholes, G. D.; MacMillan, D. W. C. Site-Selective Tyrosine Bioconjugation via Photoredox Catalysis for Native-to-Bioorthogonal Protein Transformation. Nat. Chem. 2021, 13 (9), 902-908. https://doi.org/10.1038/s41557-021-00733-y.
37. Nakane, K.; Sato, S.; Niwa, T.; Tsushima, M.; Tomoshige, S.; Taguchi, H.; Ishikawa, M.; Nakamura, H. Proximity Histidine Labeling by Umpolung Strategy Using Singlet Oxygen. J. Am. Chem. Soc. 2021, 143 (20), 7726-7731. https://doi.org/10.1021/jacs.1c01626.
38. Kim, J.; Li, B. X.; Huang, R. Y. C.; Qiao, J. X.; Ewing, W. R.; Macmillan, D. W. C. Site-Selective Functionalization of Methionine Residues via Photoredox Catalysis. J. Am. Chem. Soc. 2020, 142 (51), 21260-21266. https://doi.org/10.1021/jacs.0c09926.
39. Josephson, B.; Fehl, C.; Isenegger, P. G.; Nadal, S.; Wright, T. H.; Poh, A. W. J.; Bower, B. J.; Giltrap, A. M.; Chen, L.; Batchelor-McAuley, C.; Roper, G.; Arisa, O.; Sap, J. B. I.; Kawamura, A.; Baldwin, A. J.; Mohammed, S.; Compton, R. G.; Gouverneur, V.; Davis, B. G. Light-Driven Post-Translational Installation of Reactive Protein Side Chains. Nature 2020, 585 (7826), 530-537. https://doi.org/10.1038/s41586-020-2733-7.
40. Bloom, S.; Liu, C.; Kolmel, D. K.; Qiao, J. X.; Zhang, Y.; Poss, M. A.; Ewing, W. R.; Macmillan, D. W. C. Decarboxylative Alkylation for Site-Selective Bioconjugation of Native Proteins via Oxidation Potentials. Nat. Chem. 2018, 10 (2), 205-211. https://doi.org/10.1038/nchem.2888.
41. Singh, K.; Fennell, C. J.; Coutsias, E. A.; Latifi, R.; Hartson, S.; Weaver, J. D. Light Harvesting for Rapid and Selective Reactions: Click Chemistry with Strain-Loadable Alkenes. Chem 2018, 4 (1), 124-137. https://doi.org/10.1016/j.chempr.2017.11.007.
42. Li, J.; Kong, H.; Huang, L.; Cheng, B.; Qin, K.; Zheng, M.; Yan, Z.; Zhang, Y. Visible Light-Initiated Bioorthogonal Photoclick Cycloaddition. J. Am. Chem. Soc. 2018, 140 (44), 14542-14546. https://doi.org/10.1021/jacs.8b08175.
43. Lechner, V. M.; Nappi, M.; Deneny, P. J.; Folliet, S.; Chu, J. C. K.; Gaunt, M. J. Visible-Light-Mediated Modification and Manipulation of Biomacromolecules. Chem. Rev. 2021. https://doi.org/10.1021/acs.chemrev.1c00357.
44. Kumar, G. S.; Lin, Q. Light-Triggered Click Chemistry. Chem. Rev. 2021, 121 (12), 6991-7031. https://doi.org/10.1021/acs.chemrev.0c00799.
45. Mayer, S. V.; Murnauer, A.; von Wrisberg, M. K.; Jokisch, M. L.; Lang, K. Photo-Induced and Rapid Labeling of Tetrazine-Bearing Proteins via Cyclopropenone-Caged Bicyclononynes. Angew. Chemie-Int. Ed. 2019, 58 (44), 15876-15882. https://doi.org/10.1002/anie.201908209.
46. Yu, Z.; Pan, Y.; Wang, Z.; Wang, J.; Lin, Q. Genetically Encoded Cyclopropene Directs Rapid, Photoclick-Chemistry-Mediated Protein Labeling in Mammalian Cells. Angew. Chemie-Int. Ed. 2012, 51 (42), 10600-10604. https://doi.org/10.1002/anie.201205352.
47. Song, W.; Wang, Y.; Qu, J.; Lin, Q. Selective Functionalization of a Genetically Encoded Alkene-Containing Protein via “Photoclick Chemistry” in Bacterial Cells. J Am. Chem. Soc. 2008, 130 (30), 9654-9655. https://doi.org/10.1021/ja803598e.
48. Zhang, H.; Trout, W. S.; Liu, S.; Andrade, G. A.; Hudson, D. A.; Scinto, S. L.; Dicker, K. T.; Li, Y.; Lazouski, N.; Rosenthal, J.; Thorpe, C.; Jia, X.; Fox, J. M. Rapid Bioorthogonal Chemistry Turn-on through Enzymatic or Long Wavelength Photocatalytic Activation of Tetrazine Ligation. J. Am. Chem. Soc. 2016, 138 (18), 5978-5983. https://doi.org/10.1021/jacs.6b02168.
49. Truscott, R. J. W.; Manthey, M. K. The Oxidation of 4-Hydroxyindole as a Model System for the Oxidation of 4-Hydroxyindolyl-3-Methyl Glucosinolate. J. Sci. Food Agric. 1989, 47 (2), 191-195. https://doi.org/https://doi.org/10.1002/jsfa.2740470206.
50. Daniels, P. N.; Lee, H.; Splain, R. A.; Ting, C. P.; Zhu, L.; Zhao, X.; Moore, B. S.; van der Donk, W. A. A Biosynthetic Pathway to Aromatic Amines That Uses Glycyl-TRNA as Nitrogen Donor. Nat. Chem. 2021. https://doi.org/10.1038/s41557-021-00802-2.
51. Bauch, M.; Fudickar, W.; Linker, T. Stereoselective [4+2] Cycloaddition of Singlet Oxygen to Naphthalenes Controlled by Carbohydrates. Molecules 2021, 26 (4). https://doi.org/10.3390/molecules26040804.
52. Liu, M.; Zhang, Z.; Cheetham, J.; Ren, D.; Zhou, Z. S. Discovery and Characterization of a Photo-Oxidative Histidine-Histidine Cross-Link in IgG1 Antibody Utilizing 180-Labeling and Mass Spectrometry. Anal. Chem. 2014, 86 (10), 4940-4948. https://doi.org/10.1021/ac500334k.
53. Matsumoto, M.; Yamada, M.; Watanabe, N. Reversible 1,4-Cycloaddition of Singlet Oxygen to N-Substituted 2-Pyridones: 1,4-Endoperoxide as a Versatile Chemical Source of Singlet Oxygen. Chem. Commun. 2005, No. 4, 483-485. https://doi.org/10.1039/b414845b.
54. Martinez, G. R.; Ravanat, J. L.; Cadet, J.; Miyamoto, S.; Medeiros, M. H. G.; Di Mascio, P. Energy Transfer between Singlet (1Δg) and Triplet (3Σg-) Molecular Oxygen in Aqueous Solution. J. Am. Chem. Soc. 2004, 126 (10), 3056-3057. https://doi.org/10.1021/ja039111 z.
55. Kondo, Y.; Imai, J.; Inoue, H. Reaction of Protoberberine-Type Alkaloids. Part 12. A Facile Method for Regiospecific Oxygenation and Excited Oxidative Ring-Cleavage of Berberine Alkaloids. J. Chem. Soc.{,}Perkin Trans. 1 1980, No. 0, 911-918. https://doi.org/10.1039/P19800000911.
56. Tamura, Y.; Akita, M.; Kiyokawa, H.; Chen, L.-C.; Ishibashi, H. 1,3-Dipolar Cycloaddition of 5-Methoxy-1-Methyl-3-Oxidopyridinium. Tetrahedron Lett. 1978, 19 (20), 1751-1752. https://doi.org/https://doi.org/10.1016/0040-4039(78)80034-3.
57. Gray, M. A.; Tao, R. N.; Deporter, S. M.; Spiegel, D. A.; McNaughton, B. R. A Nanobody Activation Immunotherapeutic That Selectively Destroys HER2-Positive Breast Cancer Cells. ChemBioChem 2016, 17 (2), 155-158. https://doi.org/10.1002/cbic.201500591.
58. Meineke, B.; Heimgartner, J.; Eirich, J.; Landreh, M.; Elsasser, S. J. Site-Specific Incorporation of Two NcAAs for Two-Color Bioorthogonal Labeling and Crosslinking of Proteins on Live Mammalian Cells. Cell Rep. 2020, 31 (12). https://doi.org/10.1016/j.celrep.2020.107811.
59. Zheng, Y.; Addy, P. S.; Mukherjee, R.; Chatterjee, A. Defining the Current Scope and Limitations of Dual Noncanonical Amino Acid Mutagenesis in Mammalian Cells. Chem. Sci. 2017, 8 (10), 7211-7217. https://doi.org/10.1039/c7sc02560b.
60. Xiao, H.; Chatterjee, A.; Choi, S. H.; Bajjuri, K. M.; Sinha, S. C.; Schultz, P. G. Genetic Incorporation of Multiple Unnatural Amino Acids into Proteins in Mammalian Cells. Angew. Chemie-Int. Ed. 2013, 52 (52), 14080-14083. https://doi.org/10.1002/anie.201308137.
61. Zheng, Y.; Mukherjee, R.; Chin, M. A.; Igo, P.; Gilgenast, M. J.; Chatterjee, A. Expanding the Scope of Single- and Double-Noncanonical Amino Acid Mutagenesis in Mammalian Cells Using Orthogonal Polyspecific Leucyl-TRNA Synthetases. Biochemistry 2018, 57 (4), 441-445. https://doi.org/10.1021/acs.biochem.7b00952.
62. Chalker, J. M.; Bernardes, G. J. L.; Davis, B. G. A “Tag-and-Modify” Approach to Site-Selective Protein Modification. Acc. Chem. Res. 2011, 44 (9), 730-741.
63. Mehl, R. A.; Anderson, J. C.; Santoro, S. W.; Wang, L.; Martin, A. B.; King, D. S.; Horn, D. M.; Schultz, P. G. Generation of a Bacterium with a 21 Amino Acid Genetic Code. J. Am. Chem. Soc. 2003, 125 (4), 935-939.
64. Zambaldo, C.; Luo, X.; Mehta, A. P.; Schultz, P. G. Recombinant Macrocyclic Lanthipeptides Incorporating Non-Canonical Amino Acids. J Am. Chem. Soc. 2017, 139 (34), 11646-11649.
65. Drienovski, I.; Roelfes, G. Expanding the Enzyme Universe with Genetically Encoded Unnatural Amino Acids. Nat. Catal. 2020, 3 (3), 193-202.
66. Hartwig, J. F.; Ward, T. R. New “Cats” in the House: Chemistry Meets Biology in Artificial Metalloenzymes and Repurposed Metalloenzymes. Acc. Chem. Res. 2019, 52 (5), 1145.
67. E. D. Ficaretta, C. J. J. Wrobel, S. J. S. Roy, S. B. Erickson, J. S. Italia, A. Chatterjee, J. Mol. Biol. 2022, 434, 167304.
68. V. Perron, S. Abbott, N. Moreau, D. Lee, C. Penney, B. Zacharie, Synthesis (Stuttg). 2009, 283-289.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of chemoselective modification of a protein or peptide, wherein the protein or peptide comprises one, or more, non-canonical amino acids comprising a 5-hydroxyindole group or a 5-aminoindole group, the method comprising reacting the non-canonical amino acid of the protein or peptide with an aromatic amine derivative in the presence of a photocatalyst under conditions suitable to conjugate/covalently couple the aromatic amine derivative to the non-canonical amino acid of the protein or peptide, thereby modifying the protein or peptide in a chemoselective manner.

2. The method of claim 1, wherein the photocatalyst is reactive under visible light irradiation.

3. The method of claim 2 wherein the visible light is in the range of about 420 nm to about 650 nm.

4. The method of claim 2, wherein the photocatalyst is selected from the group consisting of: methylthioninium chloride (methylene blue), rose bengal, eyosin-Y, crystal violet, malachite green, flavin derivatives, or bromophenol blue.

5. The method of claim 1, wherein the method is externally controlled by light exposure.

6. The method of claim 1, wherein the non-canonical amino acid is chemoselectively labeled over any other canonical amino acid in the protein or peptide.

7. The method of claim 1, wherein the aromatic amine derivative comprises a cargo moiety.

8. The method of claim 7, wherein the cargo moiety comprises a detectable label.

9. The method of claim 8, wherein the label is selected from the group consisting of a dye; a fluorophore; biotin, drug molecule, polymer (e.g., PEG), or a radioisotope.

10. The method of claim 7, wherein the cargo moiety is an alkyne.

11. The method of claim 1, wherein the non-canonical amino acid comprising a 5-hydroxyindole group is 5-hydroxytryptophan (5HTP), or an analog thereof.

12. The method of claim 1, wherein the non-canonical amino acid comprising a 5-aminoindole group is 5-aminotyrtophan, or an analog thereof.

13. A method of visible light-enabled photocatalytic oxidative protein conjugation, the method comprising contacting a protein or peptide, wherein the protein or peptide comprises one, or more, non-canonical amino acids comprising a 5-hydroxyindole group or a 5-aminoindole group, with an aromatic amine derivative in the presence of a visible light enabled photocatalyst under aerobic conditions suitable to conjugate the aromatic amine derivative to the non-canonical amino acid of the protein or peptide.

14. A method of chemoselective conjugation of a protein, peptide, antibody, antibody fragment or nanobody, the method comprising:
a) incorporating one, or more, non-canonical amino acids into the protein or peptide in a site-specific manner; and
b) reacting the one, or more non-canonical amino acids with an aromatic amine derivative under photocatalytic oxidative conditions suitable to covalently conjugate the aromatic amine derivative to the non-canonical amino acid of the protein or peptide, thereby modifying the protein or peptide in a chemoselective manner.

15. The method of claim 14, wherein the non-canonical amino acid comprises a 5-hydroxyindole group or a 5-aminoindole group.

16. The method of claim 15, wherein the 5-hydroxyindole group is 5-hydroxytryptophan (5HTP), or an analog thereof.

17. The method of claim 15, wherein the 5-aminoindole group comprises is 5-aminotyrptophan, or an analog thereof.

18. A kit for the chemoselective modification of a protein or peptide, wherein the protein or peptide comprises one, or more, non-canonical amino acids, the kit comprising;
a) a container containing an aromatic amine derivative compound;
b) a photocatalyst; and
c) reagents suitable for the conjugation of the aromatic amine derivative compound to the protein or peptide.

19. The kit of claim 18, wherein the aromatic amine derivative comprises a cargo moiety.
